(19) United States
(12) Patent Application Publication
(10) Pub. No.: US 2007/0219430 A1
(43) Pub. Date: Sep. 20, 2007

(54) Electricity Providing Privately Provisioned Subscription-Based Survival Supply Unit Method And Apparatus

(76) Inventor: Barrett H. Moore, Winnetka, IL (US)

Correspondence Address: FITCH EVEN TABIN AND FLANNERY, 120 SOUTH LA SALLE STREET, SUITE 1600, CHICAGO, IL, 60603-3406, US

(21) Appl. No.: 11/539,798

(22) Filed: Oct. 9, 2006

Related U.S. Application Data
(63) Continuation-in-part of application No. 11/384,037, filed on Mar. 17, 2006.
Application No. 11/384,037 is a continuation-in-part of application No. 11/394,350, filed on Mar. 30, 2006.
Application No. 11/394,350 is a continuation-in-part of application No. 11/279,333, filed on Apr. 11, 2006.
Application No. 11/279,333 is a continuation-in-part of application No. 11/379,929, filed on Apr. 24, 2006.
Application No. 11/379,929 is a continuation-in-part of application No. 11/381,247, filed on May 2, 2006.
Application No. 11/381,247 is a continuation-in-part of application No. 11/381,257, filed on May 2, 2006.
Application No. 11/381,257 is a continuation-in-part of application No. 11/381,265, filed on May 2, 2006.
Application No. 11/381,265 is a continuation-in-part of application No. 11/381,277, filed on May 2, 2006.
Application No. 11/381,277 is a continuation-in-part of application No. 11/383,022, filed on May 12, 2006.
Application No. 11/383,022 is a continuation-in-part of application No. 11/420,594, filed on May 26, 2006.
Application No. 11/420,594 is a continuation-in-part of application No. 11/421,694, filed on Jun. 1, 2006.
Application No. 11/421,694 is a continuation-in-part of application No. 11/423,594, filed on Jun. 12, 2006.
Application No. 11/423,594 is a continuation-in-part of application No. 11/425,043, filed on Jun. 19, 2006.
Application No. 11/425,043 is a continuation-in-part of application No. 11/426,231, filed on Jun. 23, 2006.
Application No. 11/426,231 is a continuation-in-part of application No. 11/456,472, filed on Jul. 10, 2006.
Application No. 11/456,472 is a continuation-in-part of application No. 11/461,605, filed on Aug. 1, 2006.
Application No. 11/461,605 is a continuation-in-part of application No. 11/461,624, filed on Aug. 1, 2006.
Application No. 11/461,624 is a continuation-in-part of application No. 11/462,795, filed on Aug. 7, 2006.
Application No. 11/462,795 is a continuation-in-part of application No. 11/462,845, filed on Aug. 7, 2006.
Application No. 11/462,845 is a continuation-in-part of application No. 11/464,751, filed on Aug. 15, 2006.
Application No. 11/464,751 is a continuation-in-part of application No. 11/464,764, filed on Aug. 15, 2006.
Application No. 11/464,764 is a continuation-in-part of application No. 11/464,775, filed on Aug. 15, 2006.
Application No. 11/464,775 is a continuation-in-part of application No. 11/464,788, filed on Aug. 15, 2006.
Application No. 11/464,788 is a continuation-in-part of application No. 11/464,799, filed on Aug. 15, 2006.
Application No. 11/464,799 is a continuation-in-part of application No. 11/465,063, filed on Aug. 16, 2006.
Application No. 11/465,063 is a continuation-in-part of application No. 11/466,727, filed on Aug. 23, 2006.
Application No. 11/466,727 is a continuation-in-part of application No. 11/466,953, filed on Aug. 24, 2006.
Application No. 11/466,953 is a continuation-in-part of application No. 11/470,156, filed on Sep. 5, 2006.
Application No. 11/470,156 is a continuation-in-part of application No. 11/531,651, filed on Sep. 13, 2006.
Application No. 11/531,651 is a continuation-in-part of application No. 11/532,461, filed on Sep. 15, 2006.
Application No. 11/532,461 is a continuation-in-part of application No. 11/535,021, filed on Sep. 25, 2006.
Application No. 11/535,021 is a continuation-in-part of application No. 11/535,282, filed on Sep. 26, 2006.
Application No. 11/535,282 is a continuation-in-part of application No. 11/537,469, filed on Sep. 29, 2006.
(60) Provisional application No. 60/823,806, filed on Aug. 29, 2006.

(51) Int. Cl. A61B 5/00

(52) U.S. Cl. 600/300

(57) ABSTRACT

At least one transportable electric unit with survival supply items provides electrical power. Consideration-based private civil security subscriptions are accepted from subscribers with respect to providing civilly-catastrophic event-based access to the at least one transportable electric unit.

### RELATED APPLICATIONS

This application claims the benefit of U.S. Provisional Application No. 60/823,806, filed Aug. 29, 2006, which is hereby incorporated in its entirety herein.

This application comprises a continuation-in-part of each of:

SUBSCRIPTION-BASED PRIVATE CIVIL SECURITY FACILITATION METHOD as filed on Mar. 17, 2006 and having application Ser. No. 11/384,037;

SUBSCRIPTION-BASED CATASTROPHE-TRIGGERED MEDICAL SERVICES FACILITATION METHOD as filed on Mar. 30, 2006 and having application Ser. No. 11/394,350;

PERSONAL PROFILE-BASED PRIVATE CIVIL SECURITY SUBSCRIPTION METHOD as filed on Apr. 11, 2006 and having application Ser. No. 11/279,333;

RADIATION SHELTER KIT APPARATUS AND METHOD as filed on Apr. 24, 2006 and having application Ser. No. 11/379,929;

FRACTIONALLY-POSSESSED UNDERGROUND SHELTER METHOD AND APPARATUS as filed on May 2, 2006 and having application Ser. No. 11/381,247;

SUBSCRIPTION-BASED CATASTROPHE-TRIGGERED TRANSPORT SERVICES FACILITATION METHOD AND APPARATUS as filed on May 2, 2006 and having application Ser. No. 11/381,257;

SUBSCRIPTION-BASED MULTI-PERSON EMERGENCY SHELTER METHOD as filed on May 2, 2006 and having application Ser. No. 11/381,265;

SUBSCRIPTION-BASED CATASTROPHE-TRIGGERED RESCUE SERVICES FACILITATION METHOD AND APPARATUS as filed on May 2, 2006 and having application Ser. No. 11/381,277;

DOCUMENT-BASED CIVILLY-CATASTROPHIC EVENT PERSONAL ACTION GUIDE FACILITATION METHOD as filed on May 12, 2006 and having application Ser. No. 11/383,022;

RESCUE CONTAINER METHOD AND APPARATUS as filed on May 26, 2006 and having application Ser. No. 11/420,594;

PURCHASE OPTION-BASED EMERGENCY SUPPLIES PROVISIONING METHOD as filed on Jun. 1, 2006 and having application Ser. No. 11/421,694;

SUBSCRIPTION-BASED PRE-PROVISIONED TOWABLE UNIT FACILITATION METHOD as filed on Jun. 12, 2006 and having application Ser. No. 11/423,594;

RADIATION-BLOCKING BLADDER APPARATUS AND METHOD as filed on Jun. 19, 2006 and having application Ser. No. 11/425,043;

PRIVATE CIVIL DEFENSE-THEMED TELEVISION BROADCASTING METHOD as filed on Jun. 23, 2006 and having application Ser. No. 11/426,231;

EMERGENCY SUPPLIES PRE-POSITIONING AND ACCESS CONTROL METHOD as filed on Jul. 10, 2006 and having application Ser. No. 11/456,472;

PRIVATE CIVIL DEFENSE-THEMED BROADCASTING METHOD as filed on Aug. 1, 2006 and having application Ser. No. 11/461,605;

METHOD OF PROVIDING VARIABLE SUBSCRIPTION-BASED ACCESS TO AN EMERGENCY SHELTER as filed on Aug. 1, 2006 and having application Ser. No. 11/461,624;

SUBSCRIPTION-BASED INTERMEDIATE SHORT-TERM EMERGENCY SHELTER METHOD as filed on Aug. 7, 2006 and having application Ser. No. 11/462,795;

SUBSCRIPTION-BASED CATASTROPHE-TRIGGERED RESCUE SERVICES FACILITATION METHOD USING WIRELESS LOCATION INFORMATION as filed on Aug. 7, 2006 and having application Ser. No. 11/462,845;

PRIVATELY PROVISIONED SURVIVAL SUPPLIES DELIVERY METHOD as filed on Aug. 15, 2006 and having application Ser. No. 11/464,751;

PRIVATELY PROVISIONED SURVIVAL SUPPLIES SUB-UNIT-BASED DELIVERY METHOD as filed on Aug. 15, 2006 and having application Ser. No. 11/464,764;

PRIVATELY PROVISIONED SURVIVAL SUPPLIES ACQUISITION METHOD as filed on Aug. 15, 2006 and having application Ser. No. 11/464,775;

PRIVATELY PROVISIONED SURVIVAL SUPPLIES CONTENT ACQUISITION METHOD as filed on Aug. 15, 2006 and having application Ser. No. 11/464,788;

METHOD TO PRIVATELY PROVISION SURVIVAL SUPPLIES THAT INCLUDE THIRD PARTY ITEMS as filed on Aug. 15, 2006 and having application Ser. No. 11/464,799;

WASTE DISPOSAL DEVICE as filed on Aug. 16, 2006 and having application Ser. No. 11/465,063;

SUBSCRIPTION-BASED PRIVATE CIVIL SECURITY RESOURCE CUSTOMIZATION METHOD as filed on Aug. 23, 2006 and having application Ser. No. 11/466,727;

PREMIUM-BASED PRIVATE CIVIL SECURITY POLICY METHODS as filed on Aug. 24, 2006 and having application Ser. No. 11/466,953;

SUBSCRIPTION-BASED MOBILE SHELTER METHOD as filed on Sep. 5, 2006 and having application Ser. No. 11/470,156;

METHOD OF PROVIDING A FLOATING LIFE-SUSTAINING FACILITY as filed on Sep. 13, 2006 and having application Ser. No. 11/531,651;

PRIVATELY PROVISIONED SUB-UNIT-BASED SURVIVAL SUPPLIES PROVISIONING METHOD as filed on Sep. 15, 2006 and having application Ser. No. 11/532,461;

PRIVATELY PROVISIONED INTERLOCKING SUB-UNIT-BASED SURVIVAL SUPPLIES PROVISIONING METHOD as filed on Sep. 25, 2006 and having application Ser. No. 11/535,021;

RESOURCE CONTAINER AND POSITIONING METHOD AND APPARATUS as filed on Sep. 26, 2006 and having application Ser. No. 11/535,282;

PUBLICLY-FUNDED PRIVATELY FACILITATED ACCESS TO SURVIVAL RESOURCES METHOD as filed on Sep. 29, 2006 and having application Ser. No. 11/537,469;

the contents of each of which are fully incorporated herein by this reference.

### TECHNICAL FIELD

This invention relates generally to facilitating the availability of electricity for the maintenance or use of survival supply items.

### BACKGROUND

As powerful as the machinery of modern life appears, modern citizens are today perhaps more at risk of experiencing a serious disruption in their ability to prosper or even to survive en mass than is generally perceived. A serious disruption to any significant element of civilized infrastructure can produce catastrophic results for a broad swatch of a given civil community. Any number of natural and/or intentionally-caused events can significantly disrupt society's infrastructure and present a variety of differing threats and problems to the individual.

Many people believe and trust that their government (local, regional, and/or national) will provide for them in the event of such catastrophic occurrences. And, indeed, in the long view such is clearly a legitimate responsibility owed by any government to its citizens. That such is a consummation devoutly to be wished, however, does not necessarily make it so. Hurricane Katrina provided some insight into just how unprepared a series of tiered modern governmental entities may actually be to respond to even basic survival needs when a large-scale catastrophic event occurs. One may also observe that most communities simply cannot afford to staff and support a contingent capability to deal with a wide variety of likely infrequent and possibly unpredictable extreme events.

When a civilly-catastrophic event occurs, substantially sudden civil upheaval will often follow. Not only will many significant elements of society's infrastructure be seriously disrupted, but people in general are known to act in ways that are different and sometimes unexpected when faced with circumstances that threaten their well being or survival. This, coupled with the characterizing effects of the civilly-catastrophic event itself, can present an affected person with numerous challenges.

Modern governments typically do little to proactively ensure the ability to quickly and safely effect care of their citizens in the face of many civilly-catastrophic events. On the other hand, even if governments attempt to provide needed survival supply items after such catastrophic events, the catastrophic nature of the event on infrastructure may render distribution of the survival supply items difficult or impossible within a timely manner. Infrastructure such as roads, bridges, airports, and highways may be damaged or impassable rendering the transportation of such survival supply items into the affected areas impossible or time consuming at the least. Moreover, not all communities or governments have the logistical ability to correctly position survival supply items and equipment to quickly respond to an effected area. As witnessed by the effects of hurricane Katrina, it may be days or even weeks before regional, local, and national governments can immobilize and transport sufficient survival supply items into affected areas of greatest need.

Many catastrophic events are survivable presuming one has access to suitable emergency survival supply items or survival supply items, particularly during the initial clear and present threat of the event. It is possible, of course, for individuals to obtain and store such needed survival supply items in advance of any catastrophic event. Such an approach, however, presents a number of corresponding problems and issues. Attempting to take responsible actions to reasonably ensure one's own abilities in this regard can become, in and of itself, a seemingly insurmountable challenge. In addition, an individual may take the time to obtain and store emergency survival supply items, but if the supply items collected require electricity to operate or to maintain the utility of the supply items, these supply items may be rendered useless when power plants are destroyed or electrical power lines are cut during a catastrophic event. This may be especially harmful when no electricity is provided for relatively long periods of time such as days, weeks, months or more. As a result, the time and energy used to acquire and store survival supply items would be wasted.

For these and other reasons, most if not all persons are typically bereft of a suitable store of survival supply items that can be readily and quickly accessed in a time of need.

### BRIEF DESCRIPTION OF THE DRAWINGS

The above needs are at least partially met through provision of the electricity providing privately provisioned subscription-based survival supply unit method and apparatus described in the following detailed description, particularly when studied in conjunction with the drawings, wherein:

FIG. 1 comprises a flow diagram as configured in accordance with various embodiment of the invention;

FIG. 2 comprises a block diagram view as configured in accordance with various embodiments of the invention;

FIG. 3 comprises a block diagram view as configured in accordance with various embodiments of the invention;

FIG. 4 comprises a block diagram view as configured in accordance with various embodiments of the invention;

FIG. 5 comprises a block diagram view as configured in accordance with various embodiments of the invention;

FIG. 6 comprises a block diagram view as configured in accordance with various embodiments of the invention;

FIG. 7 comprises flow diagram as configured in accordance with various embodiments of the invention;

FIG. 8 comprises a flow diagram as configured in accordance with various embodiments of the invention;

FIG. 9 comprises a block diagram view as configured in accordance with various embodiments of the invention;

FIG. 10 comprises a block diagram view as configured in accordance with various embodiments of the invention; and

FIG. 11 comprises a block diagram view as configured in accordance with various embodiments of the invention.

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments of the present invention. It will further be appreciated that certain actions and/or steps may be described or depicted in a particular order of occurrence while those skilled in the art will understand that such specificity with respect to sequence is not actually required. It will also be understood that the terms and expressions used herein have the ordinary meaning as is accorded to such terms and expressions with respect to their corresponding respective areas of inquiry and study except where specific meanings have otherwise been set forth herein.

### DETAILED DESCRIPTION OF THE PREFERRED EMBODIMENT

Generally speaking, pursuant to these various embodiments, one provides at least one transportable electric unit configured to provide electrical power. Providing electrical power refers to providing any electrical device such as a source of electricity, an electrically powered survival supply device or item that is to be connected to a source of electricity, and/or an electrical conduit or conductor extending through the transportable electric unit to interconnect other electrical devices. The electric unit may also provide other non-electrical survival supply items. Consideration-based private civil security subscriptions are then accepted from one or more subscribers with respect to providing civilly-catastrophic event-based access to the electric unit.

So configured, the subscriber or corresponding authorized beneficiaries (defined below) of such consideration-based private civil security subscriptions will have predictable access to one or more electric units upon the occurrence (or threat) of a catastrophic event. In this manner, before, during or after the civilly-catastrophic event, electric units that provide electrical power (e.g. either a source of electricity or an electrically powered device) may be conveniently located for authorized beneficiaries. For instance, electric units with sources of electricity may be conveniently positioned in areas that have lost, or are most likely to lose, electric power. The electric unit with a source of electricity may also be placed at safe locations distanced from the damage or danger of the catastrophic event where little or no electricity is available. The authorized beneficiaries having access will be comforted that the type and quantity of electrical devices can be selected (and their maintenance governed) by experts and hence relieve the authorized beneficiary of responsibility in this regard.

These steps are facilitated without dependency upon governmental oversight, participation, or control. The particular type and/or quantity of survival supply item (whether or not electrical) provided can vary with the needs and requirements of the subscriber or authorized beneficiaries. Importantly, via these teachings an individual can take important steps to bring a considerably improved measure of security into their lives, knowing that, should a civilly-catastrophic event indeed be visited upon them, they will have extraordinary and reliable access to potentially needed survival supplies.

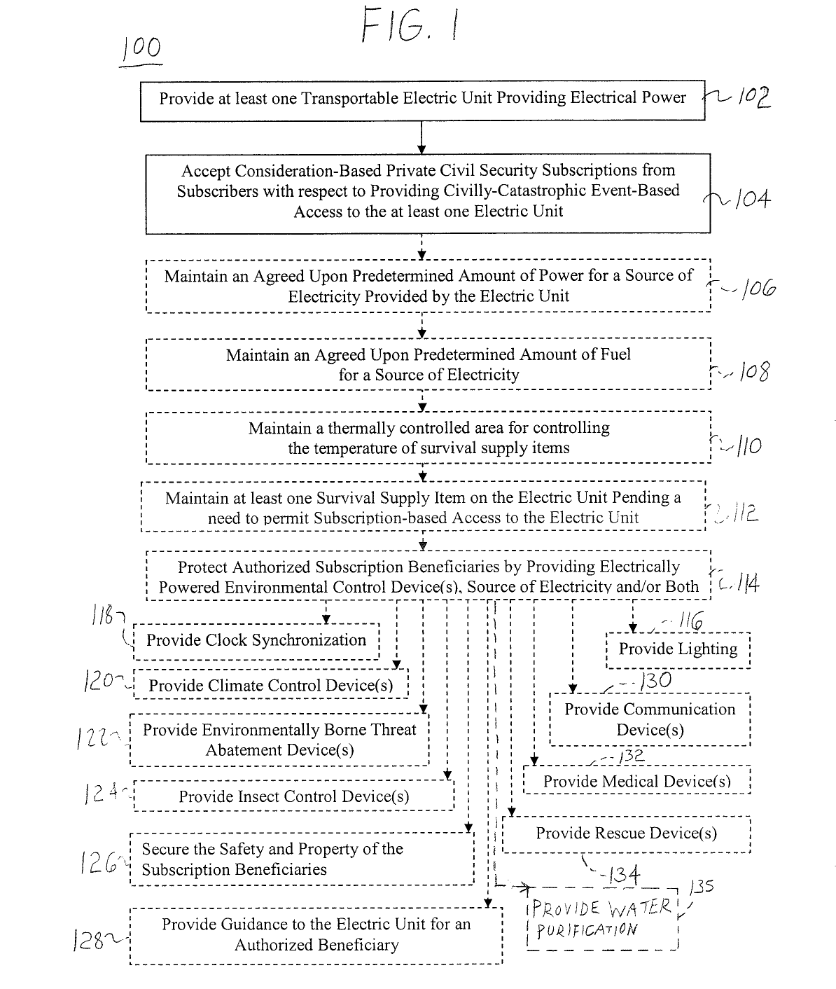

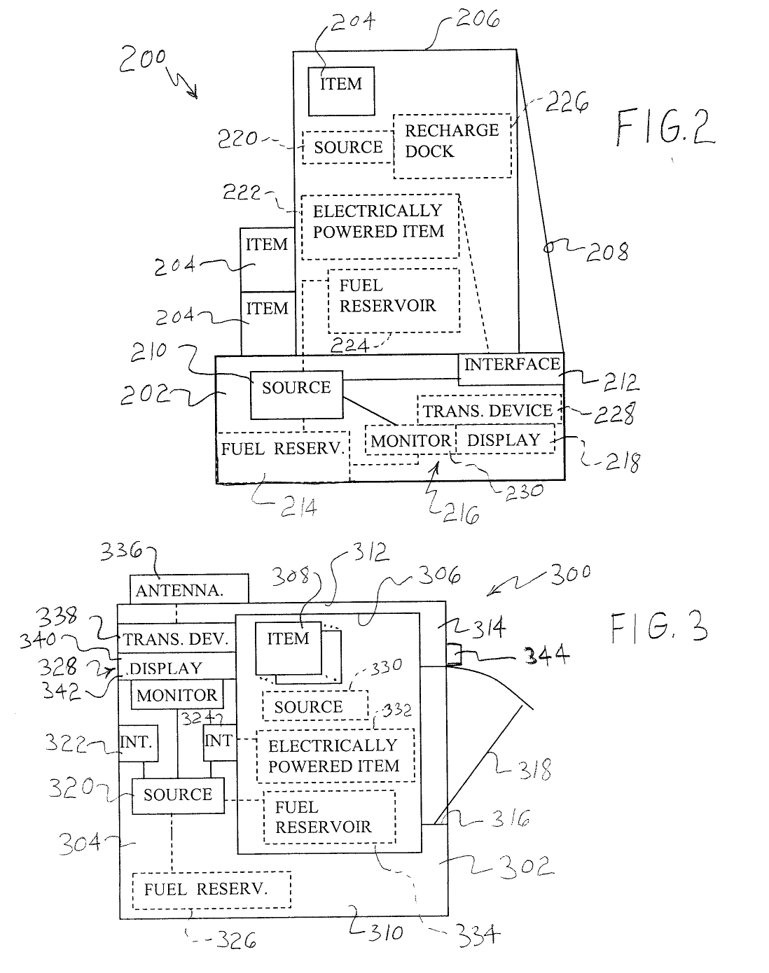

These and other benefits may become clearer upon making a thorough review and study of the following detailed description. Referring now to FIG. 1, an illustrative process 100 includes providing 102 at least one transportable electric unit configured to provide electrical power. The transportable electric unit may be any size or configuration including, but not limited to, a rectangular, cylindrical, conical, or spherical shape. As shown in FIG. 2, in one example, for certain application settings it may be appropriate that an electric unit 200 comprise a generally flat pallet 202 for holding a stack of survival supplies 204 or a container 206 itself holding survival supplies 204. Various tie-down and/or tarp or other coverings 208 can be employed in such a case to hold the survival supplies in place on the pallet. As known in the art, the pallet 202 may be made of any suitable materials including wood, plastic, or metal, and may be configured with openings, indentations, and/or additional structure to be transported by fork lifts, cranes, or any other known device for lifting loaded pallets.

The electrical devices (such as an electricity source 220, an electrically powered item 222, and a fuel reservoir 224 to name but a few) may be considered part of the survival supplies provided and may be stacked on the pallet or placed in a container 206 on the pallet. By another approach, however, the electrical devices are embedded within the pallet 202. Thus, a source of electricity 210, an electrical interface 212, a fuel reservoir 214 for the source of electricity, or monitoring equipment 216 for monitoring the source 210 and/or the fuel reservoir 214 or any combination thereof may be embedded in the pallet as is deemed appropriate. It will also be understood that any hardwire network for electricity, communication, or the like may also be embedded within the pallet 202. This configuration provides a convenient way to deliver the electrical devices while protecting the electrical devices from harmful external conditions (i.e. weather, unauthorized access, and so forth). Openings with or without movable covers may be provided for access to the electrical interface 212, a display 218 for the monitoring equipment 216, or simply to provide maintenance, removal and/or replacement of any of the embedded electrical devices. It will be appreciated that while all of the monitoring equipment 216 is shown to be embedded, only one or more components of the monitoring equipment may be embedded. It will also be understood that while the pallet 202 discloses embedding electrical devices, any survival supplies may be embedded within the pallet as is appropriate.

By another approach, as shown in FIG. 3, a transportable electric unit 300 is a container 302 formed as a fully enclosed unit with at least one wall 304 and that forms an interior, main storage space 306 for holding survival supply items 308. The container 302 is shown as rectangular but may be any other appropriate shape, and is shown with sidewall 304, a bottom wall 310, a top wall 312, and a closeable sidewall 314 with an accessway 316 covered by a door 318. If desired, the container 302 can further be configured as a lockable container as described in detail below. Alternatively, it will be understood that the container 302 may have an open top or one or more open sides instead of being a fully enclosed unit.

The electric unit 300 may carry survival items 308 and any electrical devices (such a source of electricity 330, an electrically powered item 332, or a fuel reservoir 334 for fueling a source of electricity to name a few) in the main storage space 306. As with the pallet 202, however, the container 302 may have electrical devices (and/or other survival supplies) embedded within at least one wall 304 of the container. It will be appreciated that the electrical devices may be embedded in any wall including any outer wall and even an inner partition wall if provided. Here the embedded electrical devices may include a source of electricity 320, an electrical interface 322 that faces an exterior of the container, an electrical interface 324 that faces the main storage space 306 of the container 302, a fuel reservoir 326 for supplying fuel to the source 320, and monitoring equipment 328 that monitors the fuel reservoir 326 and/or the source 320.

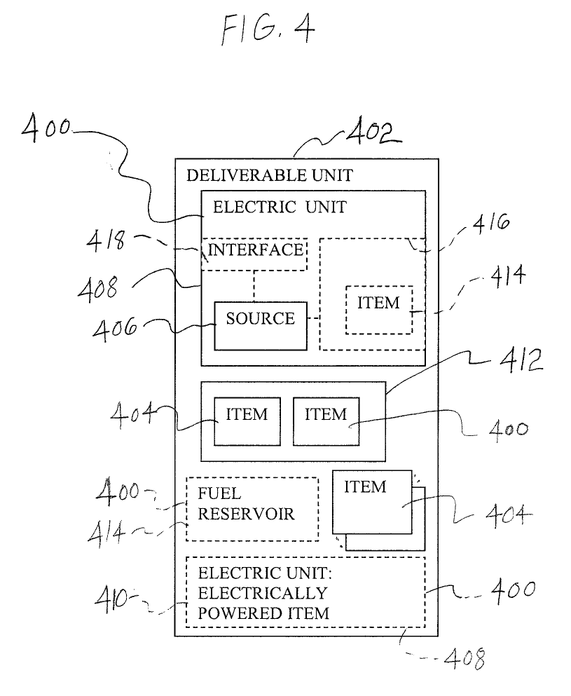

Referring to FIG. 4, in yet another approach, an electric unit 400 is considered to be at least one of the survival supply items 404 provided by a transportable or deliverable unit 402. While electric units 200 and 300 refer to an entire transportable unit and all of the supplies provided on the unit (whether electrical or not), the electric unit 400 refers only to a particular survival supply item or items provided by the deliverable unit 402. The electric unit 400 may be, or include, any of a source of electricity 406, an electrically powered device 408, monitoring equipment as mention above, and/or a fuel reservoir 410 for fueling an electricity source. Of course, the deliverable unit may also hold containers 412 that in turn hold survival supply items 400 or 404 as is appropriate.

The deliverable unit 402 has no particular shape or structure and includes any transportable body that holds one or more survival supply items 400 and/or 404. As used herein, “transportable” refers to a pallet, container, deliverable unit, or survival supply-holding body that is configured and arranged and has a size and weight such that it can be moved by a transport mechanism, such as trucks, cars, railcars, airplanes, other airships (i.e. blimps or other lighter than air aircraft), beasts of burden, ships, and boats to name but a few. For example, a common method to transport such an electric or deliverable unit would be to load the unit onto a flatbed truck or railcar by using a forklift. Alternatively, the unit may include skis, wheels, or floatation devices (or may itself be airtight to float) or may be configured and arranged to have skis, wheels, or floatation devices selectively fit thereto when needed so that the unit may be transported by being towed behind or pushed in front of a transport mechanism, such as car, truck, train, or boat.

With the structures described above, it will be appreciated that the electric unit refers to any electrically related device that assists in providing electricity. Thus, the sole purpose of the electric units may be to provide electricity (such as providing a single electrical source (such as an electrical generator), an electrically powered item (such as a refrigerator), or an electrical conduit). Alternatively, providing electricity may be one of many purposes and even a relatively insignificant purpose for providing the electric unit or deliverable unit (for example, providing a battery operated toy for entertaining children).

Turning to any of FIGS. 2-4, as mentioned above, a source of electricity may be provided by any of the electric units to power any of the electrically powered survival items provided with the electric units or electrically powered items provided separately from the electric unit but that can connect to the electric unit or the source of electricity. The source of electricity may be carried as one of the survival supply items. Optionally, the electric unit may have structure specifically configured to hold the source of electricity (also referred to herein as the “power source” or “source”) such as when the source is embedded in the wall of a container or within a pallet as previously described. Of course, the structure may be any storage structure (such as shelves, closets, drawers, bins, or the like) provided by the electric or deliverable unit.

The power source may be provided in many different forms. As is known, the power source may provide direct current (“DC”), alternating current (“AC”), or both types of current. The source may provide an inverter that inverts DC power to AC power and/or a converter that converts AC power to DC power as is known in the art. Of course, a current inverter and/or converter may be provided separately from the source as one of the electrical devices provided by the electric unit.

A specific size and type of power source may be provided to supply electrical power with a specific range of electrical characteristics in order to power specific electrically powered items. Thus, sources of electricity can be provided to supply any known range of watts, volts, and/or amperes that is needed. For instance, large residential or commercial AC generators may be provided to power one or more homes or buildings or even whole complexes or campuses. Such a generator may provide hundreds of thousands of watts. Large appliances (such as a refrigerator, microwave oven, or the like) may only need generators that supply 1000 to 10,000 watts while small appliances (such as a fax machine, fan, television receiver, lights, and so forth) may need less than 1000 watts. Power generators may be provided to supply typical appliance voltages known around the world (for example, 100-127V and 220-240V at typical frequencies (such as 50 or 60 Hertz)) or any other desired voltage). Known amperage levels (such as 15, 20, 30, 40, 100, and so forth amperes) may be provided as desired. Direct Current (DC) may also be provided in a variety of known voltages such as 1.2V (for batteries for penlights or watches for example) and higher. It will be understood that some known power sources may provide multiple selectable electrical characteristics (for example, variable wattage, voltage, and/or amperage level) so that a single power source can be adaptable to provide electricity for a variety of electrically powered devices.

In one form, the power source is at least one fuel-consumptive based source such as a generator, turbine, or micro-turbine that runs on a liquid fuel, gas fuel, fossil fuel, and/or alcohol based fuel such as gasoline, diesel, propane, or natural gas, and their substitutes such as biodiesels, straight or waste vegetable oil, or other waste oils. Alternative fuel-based sources include fuel cell sources that use hydrogen, delivered via ammonia or other known chemicals, for running clean-exhaust electric generators. Other fuels include chemical oxygen iodine electric generators that may be used for laser applications or any other appropriate application. The fuel for the fuel-consumptive based source is typically either stored in a fuel reservoir such as the reservoirs denoted by reference numerals 214, 224, 326, 334, and 414 as shown in FIGS. 2-4 as some examples. The source and electric unit may be otherwise configured to connect to an external, separately available fuel source or a fuel source on another electric unit. Any of the sources or generators described herein may be configured to remain mounted on, and movable with, the electric unit, or the sources may be detached from the electric unit and comprise a portable component that can be delivered to a convenient location. In such a case, electrical conduits may be provided to connect the source to the electric unit in order to supply electrical power to the electric unit. The fuel reservoir may be refillable.

By another approach, the source of electricity is a stored-energy based source such as batteries (but may also include or comprise fuel cells as mentioned above). The batteries may be fixed to the electric unit or loosely placed in or on the electric unit. The electric unit may be a package or container of a supply of general-purpose batteries of different sizes and voltages (For example, 12V lead acid batteries, rectangular 12 or 9 volt batteries, cylindrical A, AA, AAA, C, or D batteries, and so forth to name just a few examples). Specific-purpose batteries may also be provided such as batteries for a particular make or brand of computer, camera, communication device, or any other electronic device.

The batteries may be permanent, replaceable, or rechargeable. As shown in FIG. 2, a recharging dock 226 can be provided for electric unit 200 to detachably receive an electrical source 220 such as a rechargeable battery. While the dock 226 is shown to be provided as one of the loose survival supply items, it will be understood that the dock 226 may be embedded within the pallet, or as another example, embedded within one of the walls 304 of the container 302 of electric unit 300 (FIG. 3).

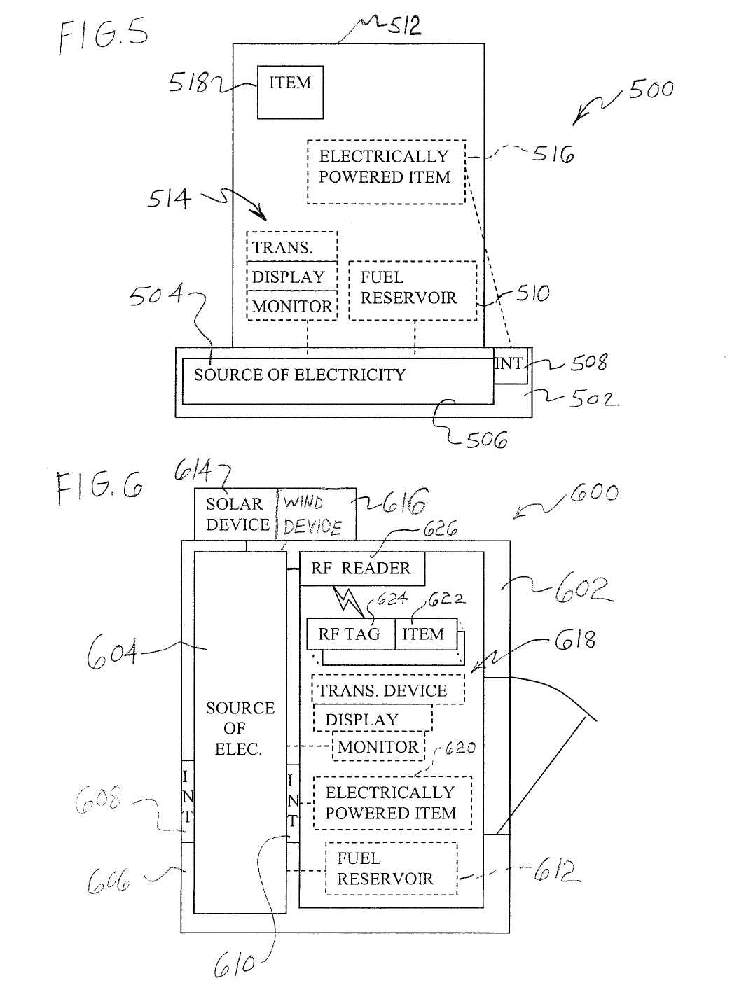

By another approach, as shown in FIG. 5, an electric unit 500 has a source of electricity 504 embedded within a pallet 502. While this source 504 may be any type of source of electricity, by one approach the source 504 is at least one rechargeable battery or fuel cell. In one form, however, the source 504 comprises hundreds or even thousands of batteries or cells. In this example form, the source 504 substantially fills an interior space 506 of the pallet 502 in order to maximize the size of source 504 and in turn the amount of energy provided by the source 504. An electrical interface 508 may be provided on or embedded within pallet 502 for electrical connection to source 504 as is known in the art, and the source 504 may be connected to a fuel reservoir 510 provided with the pallet 502 or provided separately.

As shown in FIG. 6, an electric unit 600 has a rechargeable source of electricity 604 with maximized size, similar to source 504, that may be provided on, or embedded within, a wall 606 of a container 602. The source 604 may be provided with internal or external (or both) electrical interfaces 608 and 610 for electrical connection to the source 604. A fuel reservoir 612 may also be provided to supply fuel cells on this source 604 (or other types of fuel-consumptive sources).

By yet another approach, the power source may be a photonically-based power source with a solar device 614. In one form, the solar device is the primary source of electricity. In another form, the solar device 614 is electrically connected to the rechargeable source of electricity 604 as shown on FIG. 6 for recharging the source 604. The solar device 614 may include solar panels with solar or photo cells or arrays for transforming photonic energy into electrical energy as is known. The solar panels may remain fixed against an external side of the container 602 (or the pallet 502) or may be wing-like structures configured to fold out or swing out from a side of the container 602. The solar panels may also be adjustable, whether manually or automatically, to follow a light source or solar position such as the sun for example and as known in the art. In one alternative, the solar panel device 614 may be detachable from the electric unit 600 and may have a base, stand or other configuration for mounting the solar panels on the ground or on another unit or other structure. In such a case, the solar device may be provided with appropriate power lines to connect the solar panels to a source of electricity on the electric unit or other electrical devices. With either configuration, the solar device 614 may automatically and/or continuously maintain the source 604 in a charged condition.

A wind-based power source or device 616 such as a windmill may be disposed similarly and may be the primary source of electricity or alternatively, or additionally, connected to the source of electricity 604 similarly to that of the solar device 614. For example, the wind device 616 may be detachable from the electric unit 600 for instance. Otherwise, this wind device 616 is configured with wind-to-electricity converting mechanisms as is known.

A human or animal (i.e. a “beast of burden”) powered generator could also be provided if desired. Such power generators may include a hand crank, wheel, or treadmill so that rotational motion by human or animal generates electricity as is known.

Other possible sources of electricity available include a water flow-based source (using, for example, a paddle wheel), and a thermo-electric power source (using, for example, steam-power, radiant heat power, thermoelectric materials that convert a temperature gradient directly into electricity, or the like).

By yet another approach, other known electrically powered items may be used to power the electric unit or to recharge stored-energy sources of electricity such as rechargeable batteries. Thus, the electric unit or the source of electricity may have an interface configured for electrical connection to a variety of electronic devices. The electronic device itself may be battery operated or energized by another AC power source and may have known power cables to connect to the electric unit or a rechargeable source of electricity. These electronic devices may include, but are not limited to, a communication device such as a cellular phone, a satellite phone, a computer (including but not limited to a desk top computer, a hand held computer, or a portable computer such as a laptop computer), or other electronic devices such as a camera, a watch, or the recharging docks provided with such electronic devices to name a few possible examples.

It will be understood that if multiple electric units or multiple sources of electricity are provided, any of the sources of electricity mentioned may be configured to recharge a battery type or stored-energy based source of electricity. It will also be understood that multiple sources of electricity, whether on a single electric unit or spread among multiple electric units, may be connected to the same circuit in order to combine their electrical power. Known methods may be used to configure the recharging sources to automatically recharge batteries and/or to set the sources to combine their power upon certain events such as the load on a circuit reaching a threshold amount to name one example event.

In yet another form, the electric unit provides electrically powered devices, and an external power source is coupled to the electric unit after the electric unit has been delivered to a predetermined location. For example, the electric unit could include an appropriate electrical connection such that it could be connected to a city's electrical grid via underground wiring or overhead electrical connection to power lines. Alternatively, the electric unit may be connected to any of the sources mentioned previously whether or not the source is provided by another electric unit.

In order to provide detachable connectivity between sources of electricity and electrically powered survival supply items, an electric unit may be provided with one or more electrical interfaces, such as the interface 212 shown on the electric unit 200 of FIG. 2, and the interfaces 322 and 324 shown on the electric unit 300 of FIG. 3 as a couple of examples. The electrical interfaces 212, 322 and 324 are shown to be embedded within a pallet 202 or wall 304 of a container 302 although they may be mounted on the pallet or container instead. The electrical interface may be provided to connect an electrically powered item to a source on the electric unit or vice-versa. The electrical interface may also be used to connect a second source of electricity to a rechargeable source of electricity on the electric unit or vice-versa. In one form, the interface is hard wired by an electrical conduit or conduits to the source of electricity where the interface has a distinct body from the source of electricity. For Example, the interface may comprise a separate outlet box. Alternatively, the interface may be a part of the source of electricity. In either case, conduits connecting any interfaces with electrical devices may be provided with surge protectors, power conditioners, and/or battery tenders as is known.

By one approach, the electrical interface may have any configuration that provides at least a portion of an electrical connection with at least one conductive contact configured and disposed for abutting another conductive contact of an electrical device. Thus, the electrical interface may be the plug or socket part of an outlet for a plug-socket connection with at least one prong. The outlet may provide, for instance, at least one world-wide standard AC plug or socket configuration such as one of the International Electrotechnical Commission (IEC) standard connection types A to M (where the U.S. 2-prong and 3-prong connections are types A and B respectively). In another form, the interface may also provide any one or more of a variety of DC female or male single-prong, concentric-type connections as one example.

In yet another form, the electrical connection may include at least one wire end connected to a conductor. In this case, the electrical interface may be either the wire end or the conductor. The wire end may be free or loose or may have a pre-configured end (such as loop or U-shape for example). The corresponding conductor may be a metal post, screw, terminal, or the like.

In a further form, the electrical interface may simply be at least one flat plate of conductive material to be abutted against another conductive member placed against the interface. Such a connection is typically provided for DC circuits on electronic devices and may be found with the use of rechargeable batteries. Such a connection may also be provided to electrically link electric units with each other as explained in detail below.

The electric units may also provide multiple interfaces to provide adaptability to a variety of electrical requirements from a variety of electrical devices (such as sources, powered devices, and so forth). Thus, a group of electrical interfaces may provide both alternating current and direct current. For a plug-socket connection, a group of interfaces may provide both male and female connectors and/or at least two different prong configurations. The multiple electrical interfaces may also be configured to provide at least two different electrical current characteristics such as amperes, voltage and/or wattage. For example, one interface may provide 120 volts and another interface may provide 240 volts, or any other difference as desired. It will also be understood that a single interface may provide a variation in at least one of the electrical current characteristics mentioned (such as AC, DC, amperes, voltage, or wattage) either automatically by sensing the type of device connected to the electrical interface or manually by providing a controller that controls the interface as is known.

By another approach, the electrical interface may be provided with a configuration to connect to a specific electrically powered item. In one example, the interface is configured to connect a power source on the electric unit to a vehicle or trailer (including a cart). Here vehicle refers, for example, to an automobile, a truck, a bus, a camper, a railcar, an airplane, a flying machine, a boat, a ship, a barge, a floating machine, a submersible machine, or a space travel machine, a self-balancing transporter such as a SEGWAY to note a few relevant examples. The electric unit and interface may be configured to recharge a power supply on the trailer or vehicle or may be configured to provide a continuous supply of electricity to the vehicle or trailer. Thus, the interface may be configured with, as one possible example, either the male portion or female portion of known 3-8 pin plug connections such as those known for trailers or boat dock connections. As another alternative, the electrical interface may also provide positive and negative terminals for connecting the electric unit to jumper cables that may be, in turn, connected to a vehicle with a low battery.

Referring again to FIG. 1, the process 100 also optionally includes maintaining 106 an agreed upon predetermined amount of power for a source of electricity provided by the electric unit as per the subscription agreement described in greater detail below. This maintenance 106 may be provided pending the occurrence or threat of a civilly-catastrophic event also explained in greater detail below.

Referring to FIG. 2, as an example and as mentioned above, the electric unit 200 can have embedded monitoring equipment 216 connected to source 210 for detecting the stored power level (i.e. the amount of power the source 210 has remaining). So for instance, this monitoring equipment 216 may be provided with a monitor 230 of known construction and that is connected to the source 210 as known in the art. In this case, the source 210 may be stored-energy based such as batteries or fuel cells or any other source with a detectable energy level. Once the monitor 230 detects the energy or power level of the source, this power level information may be provided to a display 218 for notifying the authorized beneficiary in possession of the electric unit 200. The display may include a display panel with various types of screens, a television, a computer having a computer monitor, a portable computer, a laptop computer, a telephone or a wireless telephone with a viewing screen, a gauge, a meter, and/or any other type of analog or digital display. An audible alert can also be provided as desired.

In addition to the power level, the displayed power information may include, but is not limited to, electrical current characteristics including at least one of: amperes, voltage, and wattage associated with the source, an amount of time remaining that the source can provide power, an amount of time the source has previously provided power, an amount of power, voltage, or amperage being used by the source, and an amount of power, voltage, or amperage being provided to the source.

In addition to, or alternative to, displaying such power level information, the information may be transmitted from a transmission device 228 on the electric unit 200 and to a party remotely monitoring the power source 210. The transmission may be accomplished automatically by data connections between the monitor and transmitter as known in the art, or the transmission may be accomplished manually by the authorized beneficiary reading the display and then communicating the power information over the transmission device 228. The party receiving the transmission may be designated by the acceptor of the subscriptions, by the subscriber, or by authorized beneficiaries of the subscriber. The recipient of the power information may be located remotely from the electric unit at a position convenient for initiating the resupply of the electric unit in order to maintain the source of electricity being monitored above a threshold power level. This may include providing further supplies to replace or repair the source of electricity. A potential type and kind of transmission device is described in greater detail below.

The designated party may also be charged with maintaining 108 the amount of fuel in the fuel reservoir that is immediately available for the source of electricity above a threshold, agreed upon level. Thus, it will be understood that instead of, or in addition to, monitoring the source of electricity 210, the monitor 230 may be monitoring the amount of fuel in the fuel reservoir 214 or 224 regardless of whether or not the reservoir is provided as one of the survival supplies (i.e. on top of the pallet 202) or embedded within the pallet 202. The power information displayed or transmitted may also include the amount of fuel remaining or consumed in order to relate how much capacity the source 210 has remaining. It will be appreciated that any of the electric units of FIGS. 2-6 may have electrical source and/or fuel reservoir monitoring equipment as described.

Referring again to FIG. 1, other than a source of electricity and associated supplies, providing 102 the electric unit for the process 100 may include providing a wide variety of other electric and non-electrical devices to assist the authorized beneficiaries before, during, and/or after the occurrence of a civilly-catastrophic event. For example, the survival supply items may be, for instance, emergency survival items, life sustaining necessities, and/or non-necessities of human life. The life's necessities may include electrical or non-electrical items such as, but not limited to, items relating to hydration (for example, water or other beverages), nourishment, shelter, environmentally borne threat abatement, medical supplies, rescue supplies, and so forth. Such survival supply items may relate to consumable items (including consumable necessities of human life), non-consumable items (including non-consumable necessities of human life), or both. Illustrative examples of consumable items would include, but are not limited to: food supplies, potable water, personal hygiene supplies, and medical supplies, to note but a few. Illustrative examples of non-consumable items could include, but are not limited to: essential clothing items, personal protection items (such as face masks, gloves, foul-weather gear, and so forth), bedding items, food preparation items, repair and maintenance tools, navigation equipment, communication items, and shelter items (such as, for example, a tent) to again note but a few.

The survival supply items may also include at least one non-necessity of human life. Illustrative examples in this regard might include, but are not limited to: a luxury consumable item, a luxury non-consumable item, non-human nourishment (for example, pet food), non-essential clothing, barter medium (including, for example, precious metals in a convenient barterable form), an entertainment apparatus or content, an educational tool, physical conditioning, exercise, and maintenance training and equipment, crafts supplies and training, and sports equipment and facilities to note but a few.

If desired, the survival supplies may also include a variety of equipment, or other survival items. For example, the survival supplies may include a transport or other vehicle as well as a vehicular fuel storage tank. In such a case, the survival supply item can also comprise, at least in part, vehicular fuel (such as, for example, gasoline, diesel fuel, and so forth) that is then pre-positioned in the vehicular fuel storage tank. Such a tank may be monitored electrically as described above for the source of electricity. By one approach, this vehicular fuel storage tank can further comprise a fuel pump such that vehicular fuel stored in the vehicular fuel storage tank can be readily transferred to the transport or other vehicle.

Also if desired, the survival supplies may be one or more spare parts of choice including, but not limited to, electrical spare parts. Such electrical spare parts might comprise, for example, spare fuses, spare illumination sources (such as light bulbs), and spare mechanical, electrical, and other maintenance parts of various types of electrical devices. The survival supplies may also provide tools for repairing or replacing any electrical device mentioned herein. There are, of course, many other examples of both electrical and non-electrical spare parts and tools that may be provided.

Referring again to FIG. 1, this process 100 may optionally include protecting 114 authorized subscription beneficiaries by providing electrically powered environmental control devices, a source of electricity, or both. By one approach, the safety of the authorized beneficiaries as well as operating some of the environmental control devices includes providing 130 a communication device. Thus, any of the electrically powered components designated reference numerals 222, 332, 410, 516, 620 (shown in FIGS. 2-6) and/or any of the transmission devices (such as transmission devices denoted as reference numerals 228 or 338 on FIGS. 2-3 respectively for example) used for monitoring a source of electricity or for monitoring any other device as explained herein may include one or more communications devices. The communication device may also be provided so that authorized beneficiaries can communicate with parties designated by the acceptor of the subscriptions, family, friends, authorities, or the like or receive news and critical information about the civilly catastrophic event and governmentally issued evacuation instructions or information as to safe locations.

The communication devices may comprise, as desired, one-way (receivers or transmitters) and/or two-way (transceiver) communication devices. A communication device may be at least partially embedded in the pallet 202 (FIG. 2) or wall 304 of such a container 302 (FIG. 3) for example, or may be provided in the main storage space 306 of a container 302 or 206 on the pallet 202. The electric unit may also provide a docking station for the communication device that is electrically connected to a source of electricity for recharging the communication device.

The communication device may be a telephone configured to be hard-wired to, or detachable from, a public or private telephone network. Otherwise, the telephone may be a short range wireless phone such as a cordless telephone or a long range wireless phone such as a cellular telephone. Other examples of communication devices include a computer with a modern such as a portable computer, a personal digital assistant (PDA) or handheld computer such as a PALM PILOT with infrared and/or BLUETOOTH linkage and the like, an email transmitting device such as a BLACKBERRY and the like, and any other Internet-linked device. Other devices include a radio transmission or receiving device (one-way or two-way devices), a citizens band (CB) radio, a short wave radio, a short-range communication device, a push-to-talk communication device (i.e. a walkie-talkie and similar devices), an image transmitting device including a camera that transmits images or video, a video relay services device, a closed-circuit television, a satellite network based-device, a microwave transmission device, a radar transmission device, a Morse code transmitter and receiver, and any combination thereof.

By one approach, a number of applications may require automatic transmissions. Thus, a number of the communication devices may be configured to transmit and/or receive data, signals, or messages automatically as is known. Optionally, other devices for automatically transmitting and/or receiving signals or data such as known transponders or transceivers may be used. In one example, these automatic transmission devices are used for the monitoring equipment 216, 328, 514, and 618 shown on FIGS. 2, 3, 5 and 6 for monitoring the source of electricity or fuel reservoirs. Automatic transmissions may also be provided for short range, wireless communication between electronic devices as well as long range communication both of which may be used, for one example, in a security system or an inventory system provided by an electric unit described in greater detail below.

Referring to FIG. 3, the electric unit 300 is shown with an exemplary antenna or antenna mast 336 operatively connected to a transmission device 338 to enhance the signal and extend the corresponding effective communications range of the transmission or communication device 338. The antenna 336 may be configured to enhance signals of a certain frequency (e.g. a certain range of radio frequencies). The antenna may be fixed on the electric unit or configured to be fixed on another structure. Thus, the antenna may be embedded within a body forming the at least one transportable electric unit, mounted on the at least one transportable electric unit, extendable from the at least one transportable electric unit, telescoping from the at least one transportable electric unit, and/or swinging from the at least one transportable electric unit. Alternatively, the antenna can be retractable and/or otherwise extendable or movable to be placed in position when needed. In another example, the antenna may have a base and an extension cable for being placed anywhere within a certain distance of the electric unit it is attached to. It will also be understood that the antenna may include known devices for manually or automatically moving and adjusting the antenna into various configurations. The antenna can further comprise, if desired, use of signal amplifiers to increase reception and/or transmission capabilities.

Referring to FIGS. 1 and 3-4, by one approach, providing 100 the electric units or electrically powered items may comprise maintaining 110 a thermally controlled area for controlling the temperature of survival supply items. As one example, electric units 300 or 400 may comprise refrigeration or an apparatus to provide refrigeration (such as a refrigerator, freezer, both, or any combination of both) for consumable survival supply items such as perishable or spoilable items, nutrients, hydration, ice, medication, medical supplies (e.g. ice packs) and any other supply that needs to be kept relatively cool or frozen. In a typical embodiment in this regard, the refrigeration will comprise, at least in part, a thermally insulated storage container such as container 302 or 408 for holding items 308 and 414 respectively. Thus, it will be understood that the electric unit may be substantially formed by a refrigerator or freezer or both (separately or as one appliance) where the sole or main purpose of the electric unit is to provide cooling. Alternatively, cooling may be provided by an electrically powered item that is one of many supplies provided by the electric unit, such as a mini-refrigerator/freezer, electrically powered cooler, or the like.

By one approach, the thermally insulated storage container is electrically powered by an external power source and can be applied via an appropriate electrical interface to any of the above described power sources. By yet another approach, the container 302 or 408 may comprise its own internal source of electricity 320 or 406, respectively, and as one example. The internal sources 320 and 406 maybe any source mentioned above. In such a case, electrically powered refrigeration could continue even during deployment of the electric units (so long as the power source remained viable). In addition, electrical interfaces 322, 324, and 418 may be provided so that other electrically powered devices can plug into the refrigeration unit to be powered by the internal sources 320 or 406.

By yet another approach, in combination with or in lieu of the approaches just mentioned, the refrigerated storage container 302 or 408 can comprise a dual-mode device that is capable of operating, for example, using propane (or a tri-mode device that is capable of automatically selecting from amongst alternating current when available, direct current when available, and propane). These and other approaches to providing refrigeration are known in the art and hence require no further detailed description here.

Maintaining a thermally controlled area may also include providing the electric unit with (or as) a heating device for cooking or providing a fire. Thus, the electric unit may provide at least one kitchen appliance such as microwave oven, electric oven, electric stove, electric pans, pots, kettles, or any other electric kitchen or camping appliance for cooking. Electric fire starting devices may also be provided.

Referring again to FIG. 1, protecting 114 authorized beneficiaries may include providing, or powering, at least one of a wide variety of environmental control devices or survival supply items. These environmental control devices are used to control the environment around authorized beneficiaries or within or around the electric unit or another designated area. Some of the broad categories these fall under are lighting, clock management, climate control, air-borne environment threat abatement, insect control, and security of persons and property to name a few. Each of these categories benefits from electrically powered devices that may be provided by or powered by the electric unit.

By one approach, protecting 114 authorized beneficiaries of the subscriptions comprises lighting 116 the exterior, the interior, or both of the at least one transportable electric unit. In one example, the electric unit has an interior portion that is automatically lit when an access way door (such as the door 318 shown on FIG. 3) is opened by use of known triggering devices. The internal lights may be manually or automatically brighter during the night to increase visibility, or instead, white light may be used during the day while red or other dim lights are manually or automatically provided during the night when keeping the electric unit hidden is a priority and/or the user's night vision is being protected. The electric unit or electrically powered devices may provide light switches of any known type for manually lighting the interior and/or exterior of the electric unit or other designated area.

Additionally, in order to light a designated area, the electric unit may provide movable lights that are detachable from the electric unit and that can be disposed a predetermined distance from the electric unit while remaining electrically connected to a source of electricity on the electric unit or elsewhere. A light switch may be provided on the light, on the electric unit, a container, and/or on the source of electricity powering the light. Further, the light(s) may be set to automatically turn on and/or off as a function, at least in part, of a time of day, detection of a certain amount of light, and detection of motion or the unauthorized unlocking, breaking or opening of an access way for security purposes as discussed below. The lights may be incandescent, gas discharging such as fluorescent or neon, light emitting diodes (LEDs), or any other light and may provide any desired range of the color spectrum as is known.

By another approach, protecting 114 authorized beneficiaries of the subscriptions comprises providing 118 the electric unit with a clock that can be synchronized with an atomic clock. This may be performed by a transmitted wireless signal received from one of the communication devices mentioned above.

By another approach, protecting 114 authorized beneficiaries may include providing 120 a climate control mechanism to control one or more of temperature, humidity, pressure, or other environmental conditions of the electric unit's interior space or another designated space. In one example, the electric units, when at least partially emptied of its contents (or when space is otherwise provided even when fully stocked), can also serve as at least a temporary shelter for one or more persons. In this regard, for many application settings the electric unit may generally comprise simplified or limited facilities suitable for accommodating a temporary stay within the electric unit for a limited number of occupants. In addition to the survival supplies already mentioned, the simplified facilities may include, for example, sleeping facilities, hygiene facilities, simple food preparation facilities, and storage facilities.

In this case, the climate control mechanism may be provided inside the electric unit or extend through walls of the electric unit when ventilation to the outside air is required for any particular climate control device. In the alternative, the climate control devices may be provided as electrically powered devices that are removable from the electric unit and that can be setup to control the climate of an interior space, such as devices placed in the window of a dwelling or building, or can be setup to change the climate of an exterior space.

For these purposes, the climate control device may be an air conditioner, a heater (whether an electrically controlled radiant heater, a space heater, or the like), a humidifier, a dehumidifier, a fan, and an electrically controlled pressure valve to name a few examples. The automatic valve may be operable on-site or remotely to equalize any pressure differential between a sealed interior space of the electric unit and any external pressure. The transportable electric unit, therefore, would be equipped to maintain a substantially constant pressure internally within the electric unit independent of any weather related low or high pressure changes. An electrical climate control panel including temperature, humidity, and pressure monitoring equipment for on-site and/or remote control may be provided and are known in the art and not described further herein.

By one approach, protecting 114 authorized beneficiaries includes protecting them from airborne contaminants and any other airborne threats. This may include providing 122 environmentally borne threat abatement devices such as filters or ionizing filters in the vicinity of an electrical fan that provides circulation to the electric unit or another enclosed space. Thus, if desired, the electric unit can optionally be configured to further include entire air mover/filter systems to thereby permit exterior filtered air to be drawn into the interior of the electric unit or other area to thereby selectively create positive air pressure relative to external ambient conditions.

By another approach, protecting 114 authorized beneficiaries may include providing 124 electrically powered environmental control devices such as electrical insect traps, insect attraction devices (such as mosquito magnets and so forth), and insect electrocutional devices (such as a “bug zapper” or the like).

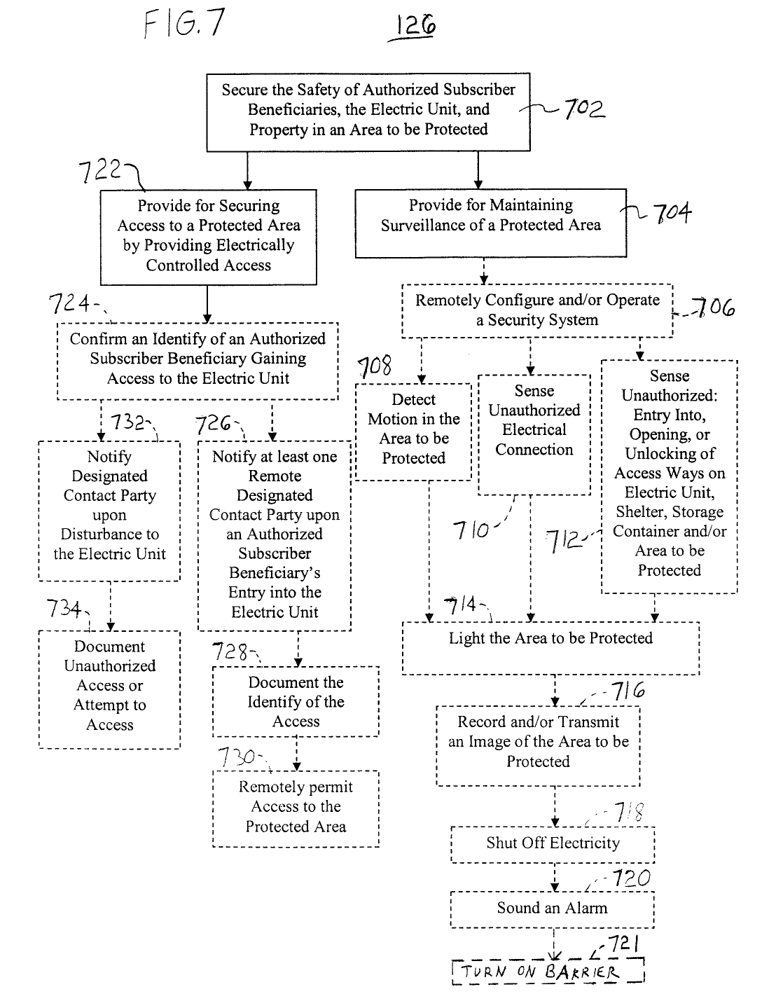

Referring now to FIGS. 1 and 7, in another form, protecting 114 authorized beneficiaries of the subscriptions includes a process 126 for securing 702 safety of the authorized beneficiaries, the at least one transportable electric unit, and/or property of the authorized beneficiaries from access by unauthorized third parties by maintaining 704 surveillance of an area to be protected. This area may be the exterior of the electric unit, the access ways into the electric unit, the area on or around secured survival items or containers, or any other designated area where the surveillance equipment supplied by or powered by the electric unit can be positioned. The property may be any survival supply item provided by the electric unit, brought to the electric unit by others, or property positioned away from the electric unit.

To perform the surveillance, the electric unit may be provided with one or more controllers as is known for configuring and operating 706 a security system or devices located at the electric unit or in another designated area to be protected. The controller may be used to turn the security system or particular components (such as an alarm or camera) of the system on or off instantaneously or set the security system and/or its components to turn on or off at certain times, or upon detection of certain events (e.g. an unauthorized entry or the like).

The controller may be remote controllers with transmission devices as described previously and as are either provided by the electric unit or positioned at other locations away from the electric unit. Thus, the remote control may be carried by any authorized beneficiary, or may be positioned at an authorized beneficiary's shelter, inside or outside the electric unit, and/or at a remote location designated by an acceptor of the subscriptions, the subscribers, or authorized beneficiaries. The designated party for maintaining a controller may be charged with the task of taking an appropriate action upon indication of events (such as an unauthorized event at the area to be protected) or of a particular condition of the security system (i.e. the security system needs maintenance).

Maintaining surveillance 704 may include the use of a security system with a wide variety of equipment. Thus, the security system may be equipped to detect motion 708 with cameras, light or air motion sensors, or other known devices. Such cameras may use infrared, microwaves, ultrasonic sound waves, or other motion detection technology as is known. Motion may also be detected by ground motion or seismic devices or by light-beam based sensors such as moving or stationary line-of-sight lights or lasers. Motion detection also includes detecting the unauthorized motion of the electric unit itself or other objects by such known devices as global positioning system devices described below or by other known sensors that detect the tilting or motion of the electric unit.

The surveillance process 704 may also include detecting 712 unauthorized entry into, opening of, and/or unlocking of an access way provided on the at least one transportable electric unit, a shelter, a storage container, and/or any other area to be protected. This may be performed by line-of-sight sensors crossing the edges of access ways or cameras pointed toward the access ways to name a few examples. In one example, the electric unit 300 has at least one accessway 316 with at least one lockable door 318. Such a door may be equipped with electronic or smart locks or other similar devices that indicate a breach as known in the art. It will be appreciated that more than one access way may be provided for the electric unit to provide multiple escape routes if needed or to provide multiple entry ways if one access way was to become impassable during or following the catastrophic event.

Maintaining surveillance 704 may also include sensing 710 unauthorized electrical connection to at least one electrical interface (322 or 324 on FIG. 3) or connection to a source of electricity 320 or 330 on the at least one transportable electric unit 300. This may be accomplished by sensing circuits as known in the art.

By one approach, the area to be protected may be constantly under surveillance, whether continuously or intermittently. Thus, when a remote control or station is used, the area to be protected may be constantly lit and a camera may be used to constantly transmit an image of the area to be protected to a remote location. The cameras that may be used for the surveillance are known and may be low-light or night-vision cameras using infrared, thermally-based, or light-intensifier based technology. In one example, the image of the exterior of the electric unit may be transmitted to closed circuit televisions viewable by the authorized beneficiaries inside the electric unit or to other designated parties anywhere in the world. The camera may also be constantly recording an image of the area to be protected, whether or not it is transmitting the image of the area to be protected to the remote controls or remote stations. The recordings may also be stored for future use and/or backed-up (e.g. duplicated for safe keeping) as is known in the art.

By another approach, upon detecting an unauthorized event, the security system is configured for lighting 714 the area to be protected by turning on general lights or alarm lights to increase visibility and to scare intruders away. The system may also be configured to activate cameras and automatically record and/or transmit 716 an image to a designated party primarily or only upon indication of an unauthorized event. The security system may also be configured to automatically shut off 718 the electricity, trigger an alarm 720, and/or turn on 721 an electric barrier and/or fence as is deemed appropriate.

Whether the security system is configured to continuously transmit the image or information of the protected area or transmit only upon detection of an unauthorized event, it will be understood that instead of operating automatically, the system may be configured for the designated recipient of the transmission to operate the security system and initiate any of the operations (e.g. triggering the alarm) mentioned above.

By another approach, additional surveillance tools may be particularly useful in determining whether or not clear and present threat exposure has been diminished or eliminated and, therefore, whether it is safe for the authorized beneficiary to approach or emerge from the transportable electric unit. In addition to the monitoring tools mentioned above, they may also comprise at least one of: an electronically operated periscope, or electrically opened or closed windows and/or shades. The monitoring equipment may also provide information related to external temperature, air quality, environmental conditions, and the scope of damage as created by the occurrence of the catastrophic event. Such information may be helpful in determining the appropriate time and direction, for example, for the occupants of the electric unit to approach or emerge from the electric unit into the external environment. When the security system or monitoring equipment is controlled remotely, the authorized beneficiary will then be able to evaluate the ability to reach the electric unit, the environmental conditions external to the electric unit, and any potential threat or other danger surrounding the electric unit safely from a remote location.

Still referring to FIG. 7, in another aspect of securing 702 the safety of authorized beneficiaries of the subscriptions and property in an area to be protected, the process 126 includes securing access 722 to a protected area, such as at least one transportable electric unit, at least one secured container, or both, by providing electrically controlled access. As mentioned above, the electric units may have a lockable access door such as the door 318 shown on the electric unit 300 for example. This access door 318 can comprise any movable-type barrier of choice including, but not limited to, a pivoting door, a sliding door, a multi-part door, and so forth. By one approach, this access door can have a corresponding locking mechanism 344. While any suitable locking mechanism can be employed in this regard including, but not limited to, key-based locks, combination locks, electronic locks, and so forth, of particular interest here, this lock 344 may comprise at least one selectively unlockable electrically or magnetically-based lock as is known. When so configured, an authorized beneficiary thereof may, if desired, be the only party having the ability to unlock the locking mechanism. More typically, however, it may be preferred to permit other authorized personnel to also have such access in order to facilitate, for example, maintenance of the electric unit and its contents. It will also be appreciated that any container, survival supply item, and electrically powered item may be provided with such locks.

The process 126 can optionally also provide for confirming 724 the authorized beneficiary's identity prior to unlocking the locking mechanism 344 to permit access through the access way or point of entry 316. The electric unit 300, therefore, may include a suitable apparatus to confirm the authorized beneficiary's identity. This may comprise, if desired, use of a mechanism that the authorized beneficiary carries with them to confirm their authorized status in this regard. This mechanism can comprise personal property (such as an identification card, a still image, an admission card) or can comprise, for example, a biometric-based identity authentication process that relies upon fingerprints or other asperity-based features, retinal patterns, voice prints, or some other relatively unique aspect of the human body or condition. In addition, the authorized beneficiary's identity may be confirmed through the use of a visual confirmation, a password, a pass code, or a word code whether keyed in, audio or otherwise.

By one approach, the confirmation may be data transmitted to designated parties that receive the transmission on known remote security control devices for such purposes. In one example, the visual confirmation is performed by transmitting a still image or video of the authorized beneficiary to a remote closed circuit television or computer. The designated party then may deny or permit access remotely.

After the authorized beneficiary's identity is confirmed and access is obtained, the process 722 then optionally provides for a notification 726 of at least one designated contact person that the authorized beneficiary has gained access to the electric unit or secured container. Such notification can occur automatically without the input or knowledge of the authorized beneficiary gaining access. The process 722 may then provide for documenting 728 the identity, time, duration, and frequency of the access for each individual authorized beneficiary that is authorized to access the electric unit. Documentation may also include documenting what survival supply item(s), if any, the authorized beneficiary uses or takes from the container. Along with such documentation, the process 722 also optionally notifies 732 the designated party of any disturbance of the electric unit (e.g. movement of the electric unit as explained above), and provides for the documentation 734 of any unauthorized attempts or actual unauthorized access. In one form, such a disturbance may include any unauthorized actions surrounding the electric unit, such as tampering, pilfering, unauthorized access, attempts at unauthorized access, unauthorized movement, attempts at unauthorized movement, or unauthorized modifications to name but a few types of disturbances. In another form, a disturbance may include damage to the electric unit that may have been sustained while experiencing the civilly-catastrophic event.

Turning back again to FIG. 1, the process 100 also optionally provides guidance 128 to the electric unit for protecting 114 the authorized beneficiaries. In one form, the guidance may include providing a predetermined location of the electric unit. Such guidance 128 will provide to the authorized beneficiary directions or coordinates to the predetermined location, and may also provide a summary of the supply or supplies provided on each electric unit provided. In this regard, depending on which survival supply item the authorized beneficiary needs, he or she can be directed to a particular predetermined location.

In one approach, the guidance 128 may be provided through at least one of globally positioning system (GPS) coordinates, an Internet communication or broadcast, a verbal communication, a written communication, a wireless communication, a visual communication or an odor-based communication to name a few. Of course, there may be other methods to provide such guidance. For example, the written communication may include the use of guide books, maps, or written directions to the predetermined locations. The visual communication may include at least one of a beacon, a strobe, a flag, a symbol, or a code (such as a color code or alphanumeric code). The odor-based communication may have an electric unit with a particular detectable odor code described to the authorized beneficiaries. The wireless communication may include at least one of a radio transmission, a television transmission, a cellular transmission, a satellite transmission, an electronic transponder signal, or a beacon signal or any other location directing signal as is known. Optionally, each separate electric unit may include a unique guidance signature based on the survival supplies provided therein.

It will also be appreciated that electronic tracking devices for GPS and transmission of other signals as mentioned or any other known tracking device may be provided permanently or removably on an electric unit. With these tracking devices, the authorized beneficiaries may be informed of their location as they are moving with the electric unit, or with the tracking device when it is removable from the electric unit.

Referring again to FIG. 1, by another approach, protecting 114 authorized beneficiaries can optionally include providing 135 a source of water or a water purification apparatus that is either monitored by a known electrical device or is purified using known electrical devices. The source of water may be provided in a water storage tank along with suitable apparatus to dispense the water (for example, pumps, valves, and so forth). Rather than a store of water, or in addition thereto, the water purification apparatus could be provided so that an authorized beneficiary would be able to cleanse water from an external source and render it potable. Various such water purification apparatus are known in the art and require no further explanation here.

By yet another approach, protecting 114 authorized beneficiaries can optionally include providing 132 medical facilities and any electrical medical supplies, equipment, and implements associated therewith. For example, the container may include medical materials to provide for a variety of medical emergencies or treatment of a variety of medical conditions. The electric unit may comprise biological system monitoring devices such as an X-ray apparatus, a magnetic resonance imaging (MRI) apparatus, an electrocardiogram (EKG) apparatus, or other equipment or other imaging platforms and tools or beds to name a few. In addition, the electric unit may comprise life sustaining or saving equipment such as a heart defibrillator, respiration device, blood circulation system devices, fluid filtration devices (for dialysis, blood, air and so forth), gas control and delivery devices (oxygen supply and associated devices for example) and surgical devices. Also, the electric unit may comprise a mobile or temporary operating or treatment room including at least some of the needed equipment, instruments, preparatory materials, and supplies to perform operations, diagnose and/or treat medical conditions, and otherwise care for patients. In this regard, an interior area of the electric unit may comprise a sterile environment or include the ability to render the interior a sterile environment when needed. This may include positive airflow filtration devices. Additionally, the electric unit may also include an electronic medical guide, which may be audio, and/or visually based, to provide instruction to the authorized beneficiary regarding how to perform medical procedures or diagnose symptoms.

By yet another approach, protecting 114 authorized beneficiaries can optionally include providing rescue 134 devices. To name one of many examples, a winch may be provided on a transportable electric unit that may be positioned to haul people, vehicles, or other objects out of danger.

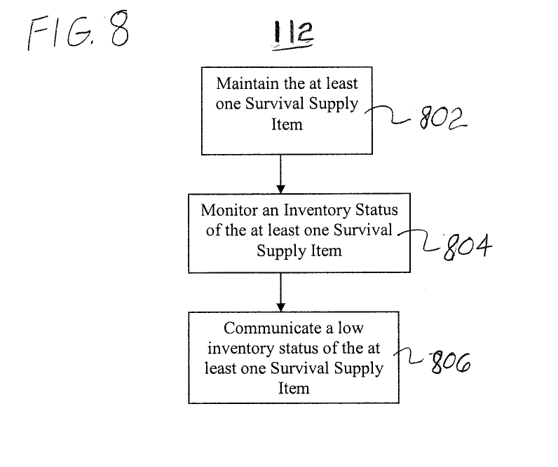

Referring to FIGS. 1 and 8, the process 100 also optionally provides for maintaining 112 the electric unit pending a need to permit subscription-based access to the electric unit upon occurrence of a catastrophic event. Such maintenance 112 can comprise, for example, maintaining both operational serviceability of the electric unit as well as maintaining 802 at least one survival supply item over time to ensure, for example, the freshness, usability, and efficacy of those supplies. Such maintenance can optionally comprise making adjustments to such supplies to reflect dynamically changing circumstances as occur during the consideration-based private civil security subscription period. As one illustration, a new item (such as a portable electrical generator as one example) may become available that is particularly useful in dealing with or otherwise surmounting some condition that may likely arise upon the occurrence of a particular kind of civilly-catastrophic event. In such a case, maintaining such supplies can readily accommodate updating the acquired and stored items to include a supply of this new item.

Accordingly, such maintenance can readily comprise one or more of removing a particular one of the items (as when a better substitute becomes available, when the item itself is shown to be less effective for its intended purpose than was originally thought, and so forth), adding additional ones of a particular item (as when it becomes subsequently understood that more of a particular item is desirable to achieve a particular goal or purpose), adding at least one new stored item that is not already stored (as illustrated in the example provided above), and so forth.

Specifically referring to FIGS. 6 and 8, in another approach, maintenance 112 may include monitoring 804 an inventory status of the at least one survival supply item. Such monitoring can include automatic monitoring such as, for example, where the electric unit 600 includes electronic monitoring devices with equipment and/or sensors such as Radio Frequency Identification (RF ID) readers 626 to automatically account for the use of, or the removal of, a particular survival item 622 that has wirelessly readable RFID tags 624 as one example system. If needed, the electric unit 600 may provide one or more sources of electricity 604 for powering an electronic network of one or more RFID readers 626.

So configured, the electric unit 600 may include an inventory system to identify the status of the survival supply items. Such status could be of only one particular survival supply item (i.e., such as the amount of water remaining) or could be a summary report of the percentage or number of each reference remaining or used as compared to an original or total quantity. Alternatively, such monitoring can include an authorized beneficiary noting on a log or other recording device (i.e., paper, computer, etc.) the use of or removal of a particular survival supply item. Such monitoring may also include communication 806 of an inventory status, such as a low inventory status, of the at least one survival supply item. The communication may occur via the Internet, phone, wireless, or other suitable communication method as described previously. In this regard, maintenance 112 may also include restocking any low inventory of the at least one survival supply item.

Referring now to FIGS. 1 and 9-11, providing 102 the at least one transportable electric unit may include providing an electric unit with an electrical conductor configured for providing at least one electrical pathway through the at least one transportable electric unit. In one form, electric units 904 each have at least one electrical conductor 910 with at least two ends where each end has an interface 912 for connection to one or more elongate electrical conductors such as an extension cord 908, another electric unit, or another electrical device such as an electricity source 902 or electrically powered device 906 as shown in FIG. 9. It will be understood that such a source 902 and device 906 may or may not be provided on electric units. It will also be understood that the electrical conductors 910 may have their interfaces 912 on other than opposite faces of the electric units and may have more than two interfaces to provide a wide variety of possible different configurations. This structure permits the formation of a continuous electrical conduit through a plurality of electric units and with a variable length and configuration depending on how the electric units are oriented and positioned relative to each other.

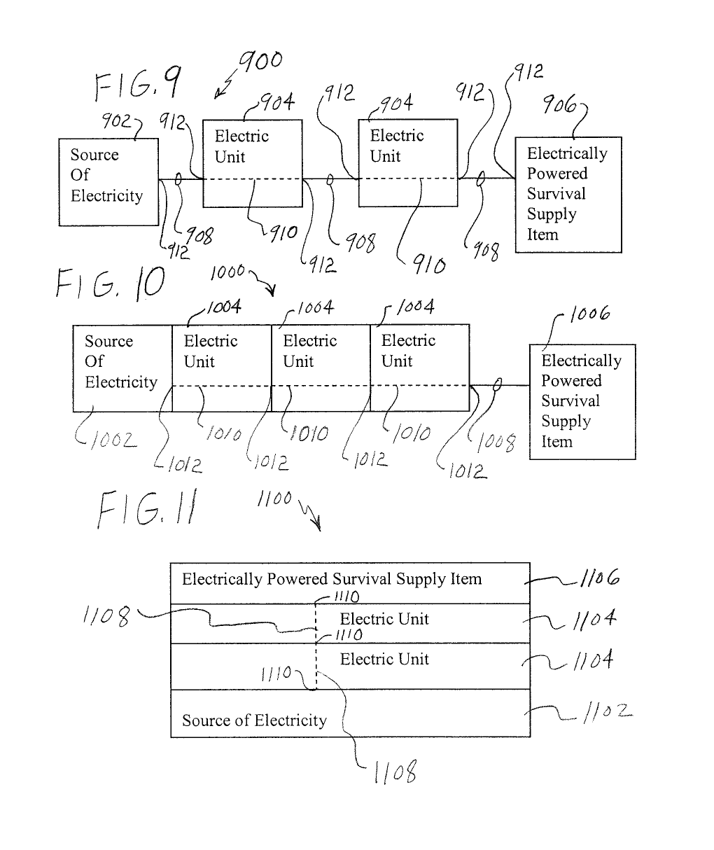

Referring to FIG. 10, by another approach, a plurality of electric units 1004 include conduits 1010 with interfaces 1012 that are configured to be placed in direct contact with at least one interface 1012 of another of the at least one transportable electric units 1004. In such a case, the interface may be flat conductive plates, plugs, or other known electrical interfaces that may be used for such purposes. Thus, the interfaces 1012 may be configured so that the transportable electric units 1004 are to be placed side to side to form the continuous electrical conduit as shown in FIG. 10. A source 1002 may be at one end of the continuously formed conduit while an electrically powered item 1006 may be connected to an electric unit 1004 by an electrical cord 1008 at a different end as one of many examples.

As shown in FIG. 11, the interfaces 1110 for corresponding conduits 1108 are configured for the transportable electric units 1104 to be stacked one upon another to form a continuous electrical conduit. In this case, a source of electrically 1102 is at the bottom of the stack while an electrically powered item 1106 may be at the top of the stack or vice-versa.

By yet another approach, an electric unit may provide one or more electrical extension cords that may or may not be selectively detachable from the electric unit. The cord(s) may also be retractably mounted on the electric unit and may have at least one end with an electrical interface fixed on the electric unit. It will be appreciated that many different configurations for a continuous conduit are possible with the use of extension cords on or between electric units, direct contact between electric units, or any combination thereof.

Again referring to FIG. 1, this process 100 provides for accepting 104 consideration-based private civil security subscriptions from subscribers with respect to providing civilly-catastrophic event-based access to at least one transportable electric unit. This right of access can pertain, if desired, to a predetermined period of time. For example, a given subscription can relate to providing access to the electric unit for a one year period of time for one or more authorized beneficiaries as correspond to that subscription.

By one approach, these subscriptions may be accepted by, for example, a for-profit business. By another approach a not-for-profit business (such as a membership-based entity) may be the appropriate entity to offer and accept such subscriptions. As noted, these teachings provide for a subscription-based approach. As used herein, the term “subscription” shall be understood to refer to and encompass a variety of legal mechanisms. Some relevant examples include, but these teachings are not limited to, subscription mechanisms such as:

time-limited rights of access (as where a subscription provides access rights for a specific period of time, such as one year, in exchange for a corresponding series of payments);

event-limited rights of access (as where a subscription provides access rights during the life of a given subscriber based upon an up-front payment in full and where those access rights terminate upon the death of the subscriber or where, for example, a company purchases a subscription for a key employee and those corresponding rights of access terminate when and if that key employee leaves the employment of that company);

inheritable rights of access (as may occur when the subscription, by its own terms and conditions, provides a right of access that extends past the death of a named subscription beneficiary and further allows for testate and/or intestate transfer to an heir);

rights of access predicated upon a series of periodic payments (as where a subscription provides access rights during, for example, predetermined periods of time on a periodic basis as where a subscriber offers month-by-month payments to gain corresponding month-by-month access rights);

rights of access predicated upon a one-time payment (as may occur when a subscriber makes a single payment to obtain a time-based or event-based duration of access rights or, if desired, when a single payment serves to acquire a one-time-only right of access or a perpetual right of access that may be retained, transferred, inherited, or the like);

ownership-based rights of access (as may occur when the subscription provides for ownership rights with respect to the survival supplies, when the subscriber comprises a stockholder of the entity that provides the resources being accessed, or the like);

non-transferable rights of access (as may occur when the subscription, by its terms and conditions, prohibits transfer of the right of access to the survival supplies from a first named beneficiary to another);

transferable rights of access (as may occur when the subscription, by its terms and conditions, permits conditional or unconditional transfer of the right of access from a first named beneficiary to another);

membership-based rights of access (as may occur when the subscription, by its terms and conditions, establishes a membership interest with respect to the accorded right of access such as, for example, a club-based membership);

fractionally-based rights of access (as may occur when the subscription, by its terms and conditions, establishes a divided or undivided co-ownership interest by and between multiple subscription beneficiaries with respect to a right to access the survival supplies);

non-ownership-based rights of access (as may occur when the subscription, by its terms and conditions, establishes the aforementioned right of access via, for example, a lease, rental, or borrowing construct); and/or

option-based rights of access.

If desired, a plurality of differentiated subscription opportunities can be offered. This plurality of differentiated subscription opportunities can correspond, for example, to providing access to differing transportable electric units, containers, and/or survival supply items. As but one very simple illustration in this regard, such subscription opportunities can differ from one another at least with respect to cost. This, in turn, provides subscriber choice with respect to selecting a particular subscription that best meets their specific needs and/or budget limitations. For example, one subscription can provide for access to a relatively large transportable electric unit that contains a relatively large store of survival and/or electrical supplies while another less expensive subscription can provide for access to a relatively smaller transportable electric unit that contains a relatively smaller store of survival and/or electrical supplies. As another example, different subscriptions can be provided that reflect different combinations and quantities of the survival and/or electrical supplies with electric units. Other possibilities are contemplated.

These teachings also readily encompass the notion of a given subscriber providing such a subscription for an authorized beneficiary other than themselves. Such might occur, for example, when one family member procures such a subscription for one or more other family members. Another example would be for a company to subscribe on behalf of named key employees, family members of such key employees, and so forth. Other examples no doubt exist.

As noted, these subscriptions relate to providing access to one or more transportable electric units providing electrical power upon the occurrence or threat of a civilly-catastrophic event. Such access may be predicated, if desired, upon a requirement that the civilly-catastrophic event be one that persists in substantial form for more than a predetermined period of time (such as one hour, one day, one week, and so forth) or that causes at least a predetermined amount or degree of infrastructure impairment or other measurable impact of choice. In addition, or in lieu thereof, such access may be predicated, if desired, upon a requirement of a particular level of objectively or subjectively ascertained likelihood that a particular category or kind of civilly-catastrophic event will occur within a particular period of time.

As used herein, “civilly-catastrophic event” will be understood to refer to an event that substantially and materially disrupts a society's local, regional, and/or national infrastructure. Such a civilly-catastrophic event can include both a precipitating event (which may occur over a relatively compressed period of time or which may draw out over an extended period of time) as well as the resultant aftermath of consequences wherein the precipitating event and/or the resultant aftermath include both the cause of the infrastructure interruption as well as the continuation of that interruption.

A civilly-catastrophic event can be occasioned by any of a wide variety of natural and/or non-naturally-caused disasters. Examples of natural disasters that are potentially capable of initiating a civilly-catastrophic event include, but are not limited to, extreme weather-related events (such as hurricanes, tsunamis, extreme droughts, widespread or unfortunately-targeted tornadoes, extreme hail or rain, and the like, flooding, and so forth), extreme geological events (such as earthquakes, volcanic activity, and so forth), extreme space-based collisions (as with comets, large asteroids, and so forth), extreme environmental events (such as widespread uncontrolled fire or the like), and global or regional pandemics, to note but a few.

Examples of non-naturally-caused disasters capable of initiating a civilly-catastrophic event include both unintended events as well as intentional acts of war, terrorism, madness or the like. Examples of non-naturally-caused disasters capable of such potential scale include, but are not limited to, nuclear-related events (including uncontrolled fission or fusion releases, radiation exposure, and so forth), acts of war, the release of deadly or otherwise disruptive biological or chemical agents or creations, exposure to a dangerous mutagenic influence, and so forth.

It would also be possible to supplement such access by permitting access to the transportable electric unit upon the occurrence of some other event or circumstance that might present the authorized beneficiary with a serious challenge while not itself necessarily rising to the level of a civilly-catastrophic event. For example, if desired, access to the subscribed-to assets might be permitted when an authorized beneficiary's home is destroyed by fire.

So configured and arranged, electrical survival supply items as may be helpful or even critical to easing the plight of authorized beneficiaries affected by a given civilly-catastrophic event are more likely to be quickly and relatively conveniently at hand. This, in turn, can greatly increase the tangible degree of civil security perceived and experienced by such persons. These teachings will readily accommodate a variety of approaches in this regard and these solutions are readily scalable to accommodate everything from relatively modest efforts to large scale prepatory activities.

Those skilled in the art will recognize that a wide variety of modifications, alterations, and combinations can be made with respect to the above described embodiments without departing from the spirit and scope of the invention, and that such modifications, alterations, and combinations are to be viewed as being within the ambit of the inventive concept.

What is claimed is:

1. A method comprising:
providing at least one transportable electric unit configured to provide electrical power; and
accepting consideration-based private civil security subscriptions from subscribers with respect to providing civilly-catastrophic event-based access to the at least one transportable electric unit.

2. The method of claim 1, wherein providing the at least one transportable electric unit comprises providing a pallet having at least one source of electricity.

3. The method of claim 2, further comprising:
providing at least one of:
(a) at least one survival supply item disposed on the pallet,
(b) at least one container holding at least one survival supply item and disposed on the pallet; and
providing a binding mechanism securing the provided one of the at least one survival supply item and the at least one container to the pallet.

4. The method of claim 2, wherein providing the at least one transportable electric unit further comprises providing an electrical interface mounted on the pallet, the electrical interface being configured for electrically connecting a survival supply item to the at least one transportable electric unit.

5. The method of claim 1, wherein providing the at least one transportable electric unit comprises providing a container with at least one wall and an interior formed within the container, the at least one transportable electric unit further comprising a source of electricity disposed at least one of:
within the interior of the container,
at least partially embedded within the at least one wall.

6. The method of claim 5, wherein the at least one wall is at least one of: an outer sidewall, a bottom wall, a top wall, and an inner partition wall.

7. The method of claim 5, wherein providing the at least one transportable electric unit further comprises providing an electrical interface mounted on the at least one wall and configured for electrically connecting an electrically powered item to the source of electricity.

8. The method of claim 1, further comprising:
providing a deliverable unit having a plurality of survival supply items, and wherein providing the at least one transportable electric unit comprises providing the at least one transportable electric unit as at least one of the survival supply items.

9. The method of claim 1, wherein the at least one transportable electric unit substantially forms a deliverable unit configured so that a sole purpose of the deliverable unit is providing electricity.

10. The method of claim 1, wherein providing the at least one transportable electric unit further comprises providing at least one source of electricity providing at least one of:
(a) direct current (DC),
(b) alternating current (AC),
(c) both alternating and direct current,
(d) an inverter that inverts DC power to AC power,
(e) a converter that converts AC power to DC power.

11. The method of claim 10, wherein the at least one source of electricity provides at least one of:
(a) multiple selectable amperage levels,
(b) multiple selectable voltage levels, and
(c) multiple selectable wattage levels.

12. The method of claim 10, wherein the at least one source of electricity is configured and disposed for being at least one of:
(a) a fuel-consumptive based source,
(b) a thermo-electric based source,
(c) a stored-energy based source,
(d) a photonically-based source,
(e) a water flow-based source,
(f) a wind-based source,
(g) human-powered based source,
(h) animal-powered based source,
(i) a portable electric generator,
(j) an electronic device,
(k) a device having at least one battery,
(l) a communication device,
(m) a cellular telephone,
(n) a satellite telephone,
(o) a computer,
(p) a portable computer,
(q) a camera,
(r) a watch.

13. The method of claim 12, wherein the fuel-consumptive based source is a generator that runs on at least one of: a liquid fuel, gasoline, diesel, a gaseous fuel, an alcohol-based fuel, propane, natural gas, biodiesel fuel, vegetable oil or waste oil.

14. The method of claim 12, wherein the fuel-consumptive based source comprises at least one fuel cell.

15. The method of claim 12, wherein the photonically-based source comprises at least one solar panel mounted on at least one of: a base and the at least one transportable electric unit.

16. The method of claim 15, wherein the at least one solar panel is at least one of: manually and automatically adjustable for following a solar position.

17. The method of claim 10, wherein the stored-energy based source comprises at least one battery that is at least one of: rechargeable, replaceable, and permanent.

18. The method of claim 1, wherein providing the at least one transportable electric unit comprises providing at least one of:
(a) at least one rechargeable battery, and
(b) a battery recharging dock configured for detachably receiving a rechargeable battery.

19. The method of claim 1, wherein providing the at least one transportable electric unit further comprises providing at least one of:
a pallet, and
a container,
wherein the provided at least one of the pallet and container generally forms an interior space substantially filled with at least one of:
at least one battery, and
at least one electricity producing fuel cell.

20. The method of claim 1, wherein providing the at least one transportable electric unit further comprises providing at least one electrical interface configured for detachable connection to at least one electrical device.

21. The method of claim 20, wherein the at least one electrical device is at least one of:
a survival supply item provided with the at least one transportable electric unit,
an electrically powered device provided separately from the at least one transportable electric unit,
a source of electricity.

22. The method of claim 20, wherein providing the at least one transportable electric unit further comprises providing a source of electricity, and wherein the at least one electrical interface is configured for electrically connecting the electrical device to the source of electricity.

23. The method of claim 20, wherein providing the at least one transportable electric unit further comprises providing at least one electrical conduit electrically connected to the at least one electrical interface.

24. The method of claim 23, wherein providing the at least one transportable electric unit further comprises providing at least one of:
(a) at least one surge protector,
(b) at least one power conditioner operatively connected to the at least one electrical conduit,
(c) at least one battery tender.

25. The method of claim 20, wherein the at least one electrical interface is adapted to be a portion of an electrical connection with at least one conductive contact configured and disposed for abutting a conductive contact of the electrical device.

26. The method of claim 25, wherein the electrical connection is at least one of:
(a) a plug-socket connection having at least one prong,
(b) a connection formed by attaching at least one free wire end onto at least one conductive member,
(c) a connection formed by attaching at least one pre-configured wire end to at least one conductive member,
(d) a connection formed by abutting at least one flat conductive contact against at least one conductive member.

27. The method of claim 20, wherein providing the at least one transportable electric unit further comprises providing multiple ones of the electrical interfaces, the multiple interfaces each comprising at least one of:
(a) both male and female connection portions,
(b) at least two different prong configurations,
(c) at least two different electrical current characteristics relating to at least one of: amperes, voltage, and wattage, and
(d) both alternating current and direct current.

28. The method of claim 20, wherein at least one electrical interface provides a plurality of selectively alternative current characteristics having a variable characteristic being at least one of: amperes, voltage, wattage, alternating current, and direct current.

29. The method of claim 20, wherein the at least one electrical interface is configured and disposed for connecting an electrical power supply to at least one of: a vehicle and a trailer.

30. The method of claim 29, wherein the at least one electrical interface is configured to connect to jumper cables.

31. The method of claim 29, wherein connecting an electrical power supply to at least one of: a vehicle and a trailer further comprises connecting to at least one of: an automobile, a truck, a camper, a railcar, an airplane, a flying machine, a boat, a ship, a barge, a floating machine, a submersible machine, a space travel machine, a cart, a self-balancing transporter.

32. The method of claim 29, wherein the electrical interface is configured for at least one of:
(a) recharging a power supply on at least one of: the vehicle and trailer,
(b) maintaining electrical power for at least one of: the vehicle and trailer.

33. The method of claim 20, wherein the electrical device is at least one rechargeable power source connected to the at least one electrical interface for detachably connecting the at least one rechargeable power source to at least one recharging power source.

34. The method of claim 33, wherein the recharging power source is at least one of:
(a) a fuel-consumptive based source,
(b) a storable-energy based source,
(c) a photonically-based source
(d) a water-flow based source,
(e) a wind-based source,
(f) a thermo-electric based source,
(g) a portable electric generator,
(h) a human-powered based source,
(i) an animal-powered based source,
(j) an electronic device,
(k) a device having at least one battery,
(l) a communication device,
(m) a cellular telephone,
(n) a satellite telephone,
(o) a computer,
(p) a portable computer,
(q) a camera,
(r) a watch.

35. The method of claim 1, wherein providing the at least one transportable electric unit further comprises providing a plurality of sources of electricity including at least one source configured to at least one of:
(a) recharge at least one of the other sources of the plurality of sources of electricity,
(b) combine its power with the power of at least one of the other sources of the plurality of sources of electricity.

36. The method of claim 35, wherein the at least one source automatically recharges the at least one of the other sources of the plurality of sources of electricity.

37. The method of claim 35, wherein the plurality of sources of electricity are provided on a plurality of electric units, and wherein the plurality of electric units are electrically connected to each other to electrically connect the at least one source to the at least one of the other sources of the plurality of sources of electricity.

38. The method of claim 1, further comprising:
maintaining an agreed upon predetermined amount of power for a source of electricity on the at least one transportable electric unit comprising:
(a) monitoring a stored power level at the source of electricity,
(b) at least one of:
(1) transmitting information including the stored power level to a party designated by an acceptor of the subscriptions, and
(2) displaying at least the stored power level on a display provided by the at least one transportable electric unit.

39. The method of claim 38, wherein the at least one transportable electric unit comprises at least one of:
(a) a pallet,
(b) a container having at least one wall, the container generally forming an interior space for holding items to be stored,
wherein the monitoring is performed by a monitor at least partially embedded within one of the pallet and the wall.

40. The method of claim 38, wherein the at least one transportable electric unit comprises at least one of:
(a) a pallet,
(b) a container having at least one wall, the container generally forming an interior space for holding items to be stored,
wherein the transmitting is performed by a transmission device at least partially embedded within one of the pallet and the wall.

41. The method of claim 38, wherein transmitting is performed by a transmission device comprising at least one of:
(a) a telephone,
(b) a wireless telephone,
(c) a cellular telephone,
(d) a computer,
(e) a portable computer,
(f) a personal digital assistant,
(g) an email transmitting device,
(h) an Internet-linked device,
(i) a radio,
(j) a citizens band radio,
(k) a short wave radio,
(l) a short-range communication device,
(m) a push-to-talk communication device,
(n) an image transmitting device,
(o) a camera,
(p) a video relay services device,
(q) a satellite network based-device,
(r) a microwave transmission device,
(s) a radar transmission device,
(t) any combination thereof.

42. The method of claim 38, wherein transmitting comprises transmitting through an antenna configured to be at least one of:
(a) embedded within a body forming the at least one transportable electric unit,
(b) mounted on the at least one transportable electric unit,
(c) extendable from the at least one transportable electric unit,
(d) telescoping from the at least one transportable electric unit,
(e) swinging from the at least one transportable electric unit.

43. The method of claim 38, wherein the at least one transportable electric unit comprises at least one of:
(a) a pallet,
(b) a container having at least one wall, the container generally forming an interior space for holding items to be stored,
wherein the displaying is performed by a display device at least partially embedded within one of the pallet and the wall.

44. The method of claim 38, wherein the displaying is performed by a display device having at least one of:
(a) a display panel,
(b) a television,
(c) a computer having a computer monitor,
(d) a portable computer,
(e) a lap-top computer,
(f) a telephone,
(g) a wireless telephone,
(h) a satellite telephone,
(i) a gauge,
(j) a meter,
(k) an analog display,
(l) a digital display.

45. The method of claim 38, wherein transmitting and displaying further comprises transmitting and displaying information relating to at least one of:
(a) electrical current characteristics including at least one of: amperes, voltage, and wattage associated with the source,
(b) an amount of time remaining that the source can provide power,
(c) an amount of time the source has previously provided power,
(d) an amount of fuel available for the source,
(e) an amount of fuel consumed by the source,
(f) an amount of power being used by the source, and
(g) an amount of power being provided to the source.

46. The method of claim 1, wherein providing the at least one transportable electric unit comprises providing a fuel reservoir configured to provide fuel for a source of electricity.

47. The method of claim 46, wherein the at least one transportable electric unit comprises at least one of:
(a) a pallet,
(b) a container with a wall,
wherein the fuel reservoir is at least partially embedded in the provided one of the pallet and the wall.

48. The method of claim 47, wherein the source of electricity is at least partially embedded within the provided one of the pallet and the wall.

49. The method of claim 47, wherein the container defines a main storage area, the method further comprising:
providing a plurality of survival supply items at least one of:
(a) on the pallet,
(b) inside the main storage area of the container,
wherein the fuel reservoir is one of the survival supply items.

50. The method of claim 49, further comprising:
maintaining at least a predetermined amount of fuel in the reservoir comprising:
monitoring the amount of fuel in the reservoir, and
at least one of:
(a) transmitting information including indication of the amount of fuel in the reservoir to a designated party associated with an acceptor of the subscriptions for refilling of the fuel reservoir,
(b) displaying the information on the at least one transportable electric unit.

51. The method of claim 1, further comprising maintaining a thermally controlled area for controlling a temperature of survival supply items.

52. The method of claim 51, wherein maintaining a thermally controlled area comprises maintaining an area that is at least one of: refrigerated and frozen.

53. The method of claim 51, wherein maintaining a thermally controlled area comprises providing the at least one transportable electric unit with at least one of:
(a) at least one source of electricity configured to connect electrically to at least one of: a refrigerator, a freezer, and a refrigerator and freezer combination,
(b) at least one of: a refrigerator, a freezer, and a refrigerator and freezer combination,
(c) a source of electricity operatively connected to at least one of: a refrigerator, a freezer, and a refrigerator and freezer combination.

54. The method of claim 53, further comprising storing at least one survival supply item of spoilable items, nutrients, hydration, medicinal items, and medical supply related items in at least one of: (a) the refrigerator, (b) the freezer, (c) the combination of (a) and (b).

55. The method of claim 51, wherein the at least one transportable electric unit is substantially formed of at least one of: (a) a refrigerator, (b) a freezer, (c) a combination of (a) and (b).

56. The method of claim 51, wherein maintaining a thermally controlled area comprises providing at least one electrical cooking device for heating consumable items.

57. The method of claim 56, wherein the at least one electrical cooking device is at least one of: a kitchen appliance, a microwave oven, an electric oven, an electric stove, and a fire starting device.

58. The method of claim 1, further comprising:
protecting authorized beneficiaries of the subscriptions by providing the at least one transportable electric unit with at least one of:
(a) at least one environmental control device that is electrically powered,
(b) at least one source of electricity configured for electrically connecting to at least one environmental control device,
(c) at least one environmental control device and at least one source of electricity operatively connected to the at least one environmental control device.

59. The method of claim 58, wherein protecting authorized beneficiaries of the subscriptions comprises lighting at least one of: an exterior and an interior of the at least one transportable electric unit.

60. The method of claim 59, wherein the at least one transportable electric unit has an interior portion and is configured to light the interior portion when the at least one transportable electric unit is at least one of: opened and occupied.

61. The method of claim 58, wherein the at least one transportable electric unit further comprises light switches for manually lighting at least one of: an interior and an exterior of the at least one transportable electric unit.

62. The method of claim 58, further comprising a light configured to be disposed a predetermined distance from the at least one transportable electric unit while remaining electrically connected to a source of electricity on the at least one transportable electric unit for lighting a selected area.

63. The method of claim 58, wherein protecting authorized beneficiaries of the subscriptions comprises setting lights to automatically turn on as a function, at least in part, of at least one of: a time of day, detection of a certain amount of light, and detection of motion.

64. The method of claim 58, wherein protecting authorized beneficiaries of the subscriptions comprises synchronizing a clock on the at least one transportable electric unit with an atomic clock.

65. The method of claim 64, further comprising receiving a transmitted wireless signal for synchronizing the clock.

66. The method of claim 58, wherein protecting authorized beneficiaries of the subscriptions includes providing the at least one transportable electric unit to be configured to at least temporarily shelter a predetermined number of the authorized beneficiaries, and wherein a climate of an interior of the at least one transportable electric unit is controlled at least in part by the at least one electrically powered environmental control device.

67. The method of claim 58, wherein protecting the authorized beneficiaries of the subscriptions comprises controlling a climate that surrounds the authorized beneficiaries by providing the at least one environmental control device as one of:
(a) an air conditioner,
(b) a heater,
(c) a humidifier,
(d) a dehumidifier,
(e) a fan,
(f) a climate control panel for controlling any of (a) to (e).

68. The method of claim 58, wherein protecting authorized beneficiaries of the subscriptions comprises protecting the authorized beneficiaries from air borne threats by providing the at least one electrically powered environmental control device with at least one electrical fan placed in a vicinity of an air filter.

69. The method of claim 58, wherein protecting authorized beneficiaries of the subscriptions comprises protecting the authorized beneficiaries from insects by providing the at least one electrically powered environmental control device with at least one of: an electrical insect trap, an insect attraction device, and an insect electrocutional device.

70. The method of claim 1, further comprising protecting authorized beneficiaries of the subscriptions including securing at least one of: safety of the authorized beneficiaries, the at least one transportable electric unit, and property of the authorized beneficiaries from access by unauthorized third parties by maintaining surveillance of an area to be protected.

71. The method of claim 70, wherein maintaining surveillance comprises
at least one of:
(a) lighting an area to be protected:
(b) transmitting an image of the area to be protected,
(c) recording an image of the area to be protected,
(d) recording and transmitting an image of the area to be protected,
(e) detecting motion in an area to be protected by using at least one of:
(1) an infrared camera,
(2) a ground movement sensor,
(3) a light beam-based sensor,
(4) a laser-based sensor.

72. The method of claim 71, wherein the image of the area to be protected is transmitted to at least one receiving device at least one of:
(a) an authorized beneficiary's location,
(b) an authorized beneficiary's shelter,
(c) inside the at least one transportable electric unit,
(d) a location remote from the at least one transportable electric unit,
(e) a remote location designated by an acceptor of the subscriptions.

73. The method of claim 70, wherein maintaining surveillance further comprises detecting motion in the area to be protected for triggering operation of at least one of:
(a) at least one camera,
(b) at least one light.

74. The method of claim 70, wherein maintaining surveillance further comprises using a low-light camera being at least one of:
(a) infrared-based,
(b) thermally-based,
(c) light-intensifier based.

75. The method of claim 70, wherein maintaining surveillance comprises sensing at least one of:
(a) unauthorized entry into,
(b) opening of,
(c) unlocking of,
an access way provided on at least one of:
(1) the at least one transportable electric unit,
(2) a shelter,
(3) a storage container.

76. The method of claim 70, wherein maintaining surveillance comprises sensing at least one unauthorized event of:
(a) unauthorized entry into the area to be protected,
(b) unauthorized movement of the at least one transportable electric unit,
(c) unauthorized electrical connection to at least one electrical interface on the at least one transportable electric unit.

77. The method of claim 76, further comprising upon detecting an unauthorized event, at least one of:
(a) transmitting indication of an unauthorized event to a designated party,
(b) triggering an alarm,
(c) activating at least one camera,
(d) turning on at least one light,
(e) shutting off the electricity to at least the at least one electrical interface,
(f) turning on an electrical barrier.

78. The method of claim 70, wherein maintaining surveillance comprises automatically transmitting an indication of an unauthorized entry to at least one of: a receiving device inside the at least one transportable electric unit and at least one remote location upon detection of the unauthorized entry.

79. The method of claim 70, wherein maintaining surveillance comprises at least one of:
(a) remotely configuring a security system provided by the at least one transportable electric unit,
(b) remotely triggering an alarm provided by the at least one transportable electric unit.

80. The method of claim 79, wherein remotely configuring and remotely triggering comprise providing at least one of:
(a) a radio frequency device,
(b) a computer,
(c) a portable computer,
(d) a wireless telephone,
(e) a satellite telephone,
(f) a cellular telephone,
for at least one of remotely:
(1) configuring the security system,
(2) triggering the security system,
(3) operating the security system.

81. The method of claim 1, further comprising protecting authorized beneficiaries of the subscriptions including protecting the safety of the authorized beneficiaries and property by electrically controlling access to at least one of:
(a) the at least one transportable electric unit,
(b) at least one secured container.

82. The method of claim 81, wherein protecting authorized beneficiaries of the subscriptions and property comprises providing at least one selectively unlockable electrically-based lock.

83. The method of claim 81, wherein protecting authorized beneficiaries of the subscriptions and property comprises confirming an authorized beneficiary's identification before permitting entry to the authorized beneficiary.

84. The method of claim 83, wherein the identity of the authorized beneficiary is confirmed electrically by at least one of:
(a) a biometric device,
(b) an identification card reading device,
(c) a camera with a transmitter configured for transmitting an image to a closed-circuit television,
(d) a pass code entry device,
(e) a password(s) identifying device.

85. The method of claim 83, wherein protecting authorized beneficiaries of the subscriptions and property comprises transmitting identification data of the authorized beneficiary to a remote security control device.

86. The method of claim 81, wherein protecting authorized beneficiaries of the subscriptions and property comprises remotely permitting access to at least one of:
(a) the at least one transportable electric unit,
(b) the secured container.

87. The method of claim 1, further comprising protecting authorized beneficiaries of the subscriptions and property including automatically notifying at least one designed contact person upon the authorized beneficiary's entry into at least one of:
(a) the at least one transportable electric unit,
(b) the secured container.

88. The method of claim 1, further comprising providing to an authorized beneficiary of one of the subscriptions guidance to the at least one transportable electric unit.

89. The method of claim 88, wherein the guidance includes at least one of: global positioning system coordinates, latitude and longitude information, a satellite communication, an Internet communication, an Internet broadcast, a verbal communication, a written communication, a wireless communication, a visual communication, an odor-based communication using an odor code.

90. The method of claim 89, wherein the visual communication includes at least one of: a beacon, a flag, a strobe, and a code.

91. The method of claim 90, wherein the code includes at least one of: a color code, a symbol, a shape, an alphanumeric code.

92. The method of claim 89, wherein the wireless communication includes at least one of: a radio transmission, a television transmission, a cellular telephony transmission, a satellite transmission.

93. The method of claim 1, wherein providing an electric unit further comprises providing at least one communication device for communicating with authorized beneficiaries of the subscriptions.

94. The method of claim 93, wherein the at least one communication device comprises:
(a) a telephone,
(b) a wireless telephone,
(c) a cellular telephone,
(d) a satellite telephone,
(e) a computer,
(f) a portable computer,
(g) a personal digital assistant,
(h) at least one of: an email receiving device and email transmitting device,
(i) an Internet-linked device,
(j) a radio,
(k) a citizens band radio,
(l) a short wave radio,
(m) a short-range communication device,
(n) a push-to-talk communication device,
(o) a Morse code-based device,
(p) an image transmitting device,
(q) a camera,
(r) a watch,
(s) any combination thereof.

95. The method of claim 93, wherein the at least one transportable electric unit provides a docking station for receiving the at least one communication device for providing electrical power to the at least one communication device.

96. The method of claim 1, further comprising enabling electrical medical emergency procedures by providing electrical medical equipment on the at least one transportable electric unit.

97. The method of claim 91, wherein the medical equipment is at least one of:
(a) a heart defibrillator,
(b) a biological system monitoring device,
(c) a respiration device,
(d) a positive airflow filtration device,
(e) a blood circulation system device,
(f) a surgical device,
(g) a fluid filtration device,
(h) a gas delivery-based device.

98. The method of claim 1, further comprising enabling electrically-based rescue operations by providing electrical rescue equipment on the at least one transportable electric unit.

99. The method of claim 98, wherein the rescue equipment at least includes a winch.

100. The method of claim 1, further comprising maintaining at least one survival supply item on the at least one transportable electric unit pending a need to permit subscription-based access to the at least one transportable electric unit.

101. The method of claim 100, wherein maintaining the at least one survival supply item comprises monitoring an inventory status of the at least one survival supply item with at least one electronic monitoring device.

102. The method of claim 101, wherein the at least one electronic monitoring device is at least one of: a radio frequency identification tag and a radio frequency identification tag reader.

103. The method of claim 102, further comprising resupplying the at least one survival supply item upon receiving a communication of a low inventory status of the at least one survival supply item.

104. The method of claim 102, wherein providing the at least one transportable electric unit comprises providing a source of electricity providing power to a network having at least one radio frequency identification tag reader.

105. The method of claim 1, wherein providing the at least one transportable electric unit comprises providing at least one survival supply item of:
(a) a life-sustaining necessity,
(b) hydration,
(c) nourishment,
(d) shelter,
(e) security,
(f) air supply devices,
(g) oxygen supply devices,
(h) environmentally borne threat abatement,
(i) medical supplies,
(j) a consumable necessity of human life,
(k) a non-consumable necessity of human life,
(l) a water purification apparatus,
(m) medical facilities and supplies associated therewith,
(n) living quarters and supplies associated therewith,
(o) a transport vehicle and supplies associated therewith,
(p) spare parts,
(q) a non-necessity of life.

106. The method of claim 1, wherein providing the at least one transportable electric unit comprises providing at least one survival supply item including at least one tool for repairing an electrical device.

107. The method of claim 1, wherein providing the at least one transportable electric unit comprises providing at least one electrical conductor configured for providing an electrical pathway through the at least one transportable electric unit.

108. The method of claim 107, wherein the electrical conductor has at least two ends, each end having an interface for connection to another electrical conductor.

109. The method of claim 108, further comprising providing a plurality of the at least one transportable electric units, and wherein at least one interface of one of the at least one transportable electric units is configured to be connected to at least one interface of any other electric unit for forming a continuous electrical conduit through at least two electric units of the plurality of electric units.

110. The method of claim 109, further comprising a plurality of electric units, and wherein the interfaces are configured to be placed in direct contact with at least one interface of another of the at least one transportable electric units.

111. The method of claim 109, wherein the interfaces are configured for the at least one transportable electric units to be placed side to side to form the continuous electrical conduit.

112. The method of claim 109, wherein the interfaces are configured for the at least one transportable electric units to be stacked one upon another to form the continuous electrical conduit.

113. The method of claim 109, further comprising providing a plurality of electric units configured to be electrically connected to each other by elongate conductive members configured to connect to the interfaces.

114. The method of claim 1, wherein providing the at least one transportable electric unit further comprises providing an extension cord on the at least one transportable electric unit.

115. The method of claim 114, wherein the extension cord is retractably mounted on the at least one transportable electric unit.

116. The method of claim 114, wherein the extension cord is selectively detachable from the at least one transportable electric unit.

117. A transportable electric unit for providing survival supply items, comprising at least one of:
(a) at least one source of electricity,
(b) at least one electrically powered device,
(c) at least one conductive conduit extending through the at least one transportable electric unit, and
consideration-based private civil security subscriptions from subscribers with respect to providing civilly-catastrophic event-based access to the at least one transportable electric unit.

118. The at least one transportable electric unit of claim 117, wherein the source of electricity comprises at least one battery.

119. The at least one transportable electric unit of claim 118, further comprising a monitor for monitoring at least an amount of power that the at least one battery has remaining.

120. The at least one transportable electric unit of claim 117, further comprising a fuel reservoir for providing fuel for a source of electricity.

121. The at least one transportable electric unit of claim 120, further comprising a monitor for monitoring an amount of fuel in the fuel reservoir for determining how much capacity the source of electricity has remaining.

122. The at least one transportable electric unit of claim 120, wherein the fuel reservoir is refillable.

123. The at least one transportable electric unit of claim 120, wherein the fuel reservoir is configured and arranged for being an external fuel supply for at least one other electric unit.

124. The at least one transportable electric unit of claim 117 being further configured and arranged to connect to an external fuel reservoir.

125. The at least one transportable electric unit of claim 117, further comprising a display device provided by the at least one transportable electric unit for displaying an amount of capacity the source of electricity has remaining.

126. The at least one transportable electric unit of claim 117, further comprising a transmission device for transmitting information indicating a condition of the source of electricity for resupplying the at least one transportable electric unit to operate the source of electricity.

127. The at least one transportable electric unit of claim 117, wherein the source of electricity comprises at least one solar panel mounted on at least one of: a base and the at least one transportable electric unit.

128. The at least one transportable electric unit of claim 127, wherein the solar panel is at least one of: manually and automatically adjustably mounted for following a light source.

129. The at least one transportable electric unit of claim 117, wherein the source of electricity comprises at least one of:
(a) a fuel-consumptive source of electricity,
(b) a wind-based source of electricity,
(c) a photonically-based source of electricity,
(d) a fluid-based source of electricity,
(e) a thermoelectric source of electricity,
(f) a stored-energy source of electricity,
(g) human-powered based source of electricity,
(h) animal-powered based source of electricity,
(i) a portable electric generator,
(j) an electronic device,
(k) a device having at least one battery,
(l) a communication device,
(m) a cellular telephone,
(n) a satellite telephone,
(o) a computer,
(p) a portable computer,
(q) a camera,
(r) a watch.

130. The at least one transportable electric unit of claim 117, wherein the at least one electrically powered device comprises a communication device.

131. The at least one transportable electric unit of claim 130, wherein the communication device is at least one of:
(a) a telephone,
(b) a wireless telephone,
(c) a cellular telephone,
(d) a satellite telephone,
(e) a computer,
(f) a portable computer,
(g) a personal digital assistant,
(h) at least one of an email transmitting device and email receiving device,
(i) an internet-linked device,
(j) a radio,
(k) a citizens band radio,
(l) a short wave radio,
(m) a short-range communication device,
(n) a push-to-talk communication device,
(o) a Morse-code-based communication device,
(p) an image transmitting device,
(q) a camera,
(r) a video relay services device,
(s) a satellite network based-device,
(t) any combination thereof.

132. The at least one transportable electric unit of claim 117, further comprising at least one survival supply item carried by the electric unit, the at least one survival supply item comprising at least one of:
(a) a life-sustaining necessity,
(b) hydration,
(c) nourishment,
(d) shelter,
(e) security,
(f) air supply devices,
(g) oxygen supply devices,
(h) environmentally borne threat abatement,
(i) medical supplies,
(j) a consumable necessity of human life,
(l) a non-consumable necessity of human life,
(l) a water purification apparatus,
(m) medical facilities and supplies associated therewith,
(n) living quarters and supplies associated therewith,
(o) a transport vehicle and supplies associated therewith,
(p) spare parts,
(q) a non-necessity of life.

133. The at least one transportable electric unit of claim 117, wherein the at least one electrically powered device comprises at least one of:
(a) a dock configured for receiving a rechargeable source of electricity,
(b) at least one of: (1) a refrigerator, (2) a freezer, (3) a combination of both (1) and (2),
(c) a climate control device for controlling climate of a designated area,
(d) a light,
(e) a clock synchronization device,
(f) an environmental air borne threat abatement device,
(g) an insect control device,
(h) a security surveillance device,
(i) a security lock device,
(j) a subscriber identification detection device,
(k) a subscriber guidance device,
(l) a medical device,
(m) a rescue device,
(n) an inventory monitoring device.
